(19) United States
(12) Patent Application Publication
(10) Pub. No.: US 2007/0099823 A1
(43) Pub. Date: May 3, 2007

(54) Inhibitors of sporeforming pathogens and applications of the same

(75) Inventors: Olga M. Tarasenka, Little Rock, AR (US); Kalle M. Levon, Brooklyn, NY (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Board of Trustees of the University of Arkansas, Little Rock, AR (US)

(21) Appl. No.: 11/261,400

(22) Filed: Oct. 28, 2005

(51) Int. Cl. A61K 38/16; A61K 31/739

(52) U.S. Cl. 514/8; 514/54

(57) ABSTRACT

An inhibitor of sporeforming pathogens or agents. In one embodiment, the inhibitor has at least one glycoconjugate that is bondable to a sporeforming pathogen or agent. The glycoconjugate is a molecule containing a carbohydrate moiety that is glycosylated. The glycosylated carbohydrate moiety may comprise a glycoprotein or a glycolipid. In one embodiment, the glycosylated carbohydrate moiety comprises at least one disaccharide moiety or higher polysaccharide. In another embodiment, the glycosylated carbohydrate moiety comprises a plurality of copies of same monosaccharide units or different monosaccharide units.

### CROSS-REFERENCE TO RELATED PATENT APPLICATION

This application claims the benefit, pursuant to 35 U.S.C. §119(e), of provisional U.S. patent application Ser. No. 60/622,862, filed Oct. 28, 2004, entitled “INHIBITORS OF SPOREFORMING PATHOGENS,” by Olga Tarasenko et al., which is incorporated herein by reference in its entirety.

Some references, if any, which may include patents, patent applications and various publications, are cited and discussed in the description of this invention. The citation and/or discussion of such references is provided merely to clarify the description of the present invention and is not an admission that any such reference is “prior art” to the invention described herein. All references, if any, cited and discussed in this specification are incorporated herein by reference in their entireties and to the same extent as if each reference was individually incorporated by reference. In terms of notation, hereinafter, “[n]” represents the nth reference cited in the reference list. For example, [179] represents the 179th reference cited in the reference list, namely, Tarasenko O., Islam Sh., Paquiot D., and Levon K. Carbohydrate Research. 2004; 339: 2859-2870.

### CROSS-REFERENCE TO RELATED PATENT APPLICATION

This application claims the benefit, pursuant to 35 U.S.C. §119(e), of provisional U.S. patent application Ser. No. 60/622,862, filed Oct. 28, 2004, entitled “INHIBITORS OF SPOREFORMING PATHOGENS,” by Olga Tarasenko et al., which is incorporated herein by reference in its entirety.

### FIELD OF THE INVENTION

The present invention is generally related to an inhibitor of sporeforming pathogens or agents, and, more particularly, is related to an inhibitor has at least one glycoconjugate that is bondable to a sporeforming pathogen or agent.

### BACKGROUND OF THE INVENTION

Sporeforming agents are responsible for a plethora of human and animal infections, contamination of food, biological and environmental samples, and buildings. Spore is dormant cell type. In particular, dormant spores of Bacillus genus are generally resistant to a variety of conditions [58, 167, 168, 35] that threaten to destroy their vegetative cell counterparts [58, 178, 210]. It was observed that spores are resistant to a variety of treatments, including heat, UV radiation and oxidizing agents [58, 178, 210, 35]. In addition, disinfectants/biocides (e.g., sodium hypochlorite, formaldehyde and phenols) that are highly effective against Bacillus spores, are not well suited for decontamination of the environment, equipment, or casualties. This is due to toxicity that leads to tissue necrosis and severe pulmonary injury following inhalation of volatile fumes. The corrosive nature of these compounds also renders them unsuitable for decontamination of sensitive equipment [1, 15, 43, 126, 144, 166]. B. anthracis, the etiologic agent of anthrax, is a large, gram-positive, rod-shaped, non-motile, facultative anaerobic, sporeforming bacterium that causes disease in humans and herbivore animals [7, 123].

The longevity [167, 168] of spores in the environment is an important factor in the epidemiology of anthrax and explains the predominant occurrence of the disease in herbivores [185]. Contamination of farmlands with B. anthracis leads to a fatal disease in domestic, agricultural, and wild animals [29]. Animal anthrax infection still represents a significant problem due to the difficulty in decontamination of land and farms.

Spore grows vegetatively exclusively in the mammalian host where spores germinate in the presence of favorable conditions such as aminoacids, sugars, adequate pH, and a favorable temperature [78, 190].

Humans acquire the disease incidentally by contact with infected animals or animal products [206] and recently as biowarfare and bioterrorism threats [106, 165]. The incidence of disease has decreased dramatically in developed countries as a result of animal vaccination programs and improved industrial hygiene. The portals of entry of bacterial spores are predominantly the skin, lungs and gastrointestinal tract.

Clinical anthrax in humans presents in three distinct forms: cutaneous, gastrointestinal, and inhalational. Eschar formation and edema at the site of inoculation characterize cutaneous anthrax. Gastrointestinal anthrax has never been reported in this country. Inhalational anthrax results from inhalation of B. anthracis spores. Inhalational anthrax progresses rapidly from nonspecific symptoms to death in the majority of cases. The gastrointestinal and cutaneous forms of anthrax, although less rapid, can result in fatalities unless treated aggressively [50, 152]. Additionally, other members of the Bacillus genus and other sporeforming pathogens are also reported to be etiological agents for many human diseases. B. cereus is a common pathogen. B. cereus is involved in food borne diseases due to the ability of the spores to survive cooking procedures. It is also associated with local sepsis and wound and systemic infection [40].

B. cereus, B. anthracis, B. thuringiensis species, along with B. mycoides, have been known to belong to the phylogenetically similar B. cereus group [163, 155]. B. anthracis exhibits genetic similarities with B. cereus [163, 155]. Indeed many consider them to be the same species [187, 82].

The first step in infections in mammalians cause by spores is generally attachment or colonization of organisms. Their immune system recognizes invading spores and quickly mobilizes the best defenders to search out and destroy these invaders such as spore. Macrophages engulf and consume spores, carry them into lymph nodes. En route, or in the macrophage the spores transform into dividing vegetative cells. Later bacterial cells erupt from macrophage and infiltrate the body, follow by toxins dissemination and disease symptom and syndrome appearance. Usually disease symptoms and syndromes appear within 7 days. The anthrax toxin is produced from B. anthracis [41]. The three components of anthrax toxin, protective antigen (PA), lethal factor (LF), and edema factor (EF), are not harmful individually, but in combination form two different pathogenic responses [175, 53].

In addition, there is concern about human infection brought about by warfare and/or terrorist activities. The potential consequences of the use of B. anthracis spores as a biological weapon was demonstrated by the accidental release of B. anthracis from a military microbiology laboratory in the former Soviet Union. Seventy-seven cases of human anthrax, including 66 deaths, were attributed to the accident. Some anthrax infections occurred as far as 4 kilometers from the laboratory [139]. Genetic analysis of infected victims revealed the presence of either multiple strains or a genetically altered B. anthracis [81]. The anthrax attacks of 2001 have increased concerns that “weapons grade” biological agents can be obtained or manufactured and disseminated by terrorists or terrorist groups. These events brought the issues surrounding the deliberate release of biological warfare agents (BWA) into sharp focus in the USA and around the globe. Due to the highly fatal nature of pulmonary anthrax (80-90%), easy of production and storage of the spores of Bacillus genus and their survival in the environment after bioattack, this organism has become the primary bacterial agent in biowarfare and bioterrorism [106, 165]. Progress has been made in the improvement of preparedness against bioterrorism attack, medical response and public health management, in the development of rapid disease diagnosis and treatment strategies, vaccination and antibiotic use; in the improvement of detection devices; and in the preparation of emergency services [106, 165, 188, 88, 67, 26, 17, 128]. To counter the threat of terrorist attacks, an effective earlier defense is required to minimize the consequences of biological attacks.

Another source of spores may be contaminated effluents from plants manufacture working with animal products, e.g. leather tanneries. Other routes of transmission have been described, e.g. biting flies or contaminated feces [47, 34, 190]. It is not only humans that are susceptible to infectious agents such as anthrax. The animals and plants [164] on which people depend also constitute potential targets for those capable of using BWA as weapons. In particular, livestock (which are natural hosts for B. anthracis) are believed to be far more vulnerable to anthrax infection [77]. Contamination of food supply could severely affect both military as well as civilian public health. Sporeforming pathogens have been responsible for the spoilage of canned foods, bread vacuum packed meat products, pasteurized dairy products, fruit juices, fruits and vegetables [10, 92, 93, 110, 65, 136, 134].

Treatment of infection usually starts after symptoms and syndromes appearance. Penicillin is the drug of choice for the treatment of anthrax infections [184]. Other acceptable alternatives include ciprofloxacin and doxycycline [184]. Although an invaluable advance, antibiotic and antimicrobial therapy suffers from several problems, particularly when strains of various bacteria appear that are resistant to antibiotics. Many bacteria readily develop resistance to antibiotics. Mutations are constantly affecting life in the bacterial world. Changes of one nucleotide (mutation) in a base pair of amino acids during a mutation can cause a structure alteration [201, 30, 31]. Mutation mechanisms have been studied in some detail in eukaryotes [201, 30, 31]. For instance, the mutation rate contributes to the adaptation of the pathogen to its hosts or environment [58]. Mutation rate in eukaryotes appears to be insensitive to mismatch repair efficiency as well. Internal heterogeneity is compatible with a high mutation rate [31]. In such an extreme situation, mutation limited a value for strain identification, epidemiological, phylogenetic and other studies, as well as drug treatments. An organism infected with an antibiotic-resistant strain of bacteria faces serious and potentially life-threatening consequences.

Whereas early treatment with antibiotics can halt progression of infection, vaccination remains the preferred method for prevention of infection and eradication of the agent [70, 186, 189]. Vaccination is the most cost effective form of mass protection. Vaccines protect against disease by harnessing the body's innate ability to protect itself against foreign invading agents; During vaccination, the patient is injected with antigens, or other encoding antigens, which generate protective antibodies but which typically can not cause severe disease themselves. For example, vaccination for bacterial diseases such as typhoid fever consists of injecting a patient with the bacterial agents of these diseases, after they have been disabled in some fashion to prevent them from causing disease. The patient's body recognizes these bacteria nonetheless and generates an antibody response against them. It is not always possible, however, to stimulate antibody formation merely by injecting the foreign agent which causes the disease. The foreign agent must be immunogenic, that is, it must be able to induce an immune response. Certain agents such as tetanus toxoid are innately immunogenic, and may be administered in vaccines. Other clinically important agents are not immunogenic, however, and must be converted into immunogenic molecules before they can induce an immune response. Successfully accomplishing this conversion for a variety of antigens is a major goal of a great deal of immunologic research. However, researchers have yet to successfully convert a variety of poorly immunogenic antigens into optimally immunogenic molecules. One approach which researchers have taken to enhance the immune response to T-independent antigens is to inject subjects with polysaccharide or oligosaccharide antigens that have been conjugated to a single thymus-dependant (T-dependent) antigen such as tetanus or diphtheria toxoid [96, 171, 3]. These conjugate vaccines improve on vaccines based on carbohydrates alone because they “trick” the T-cells into directing the immune response, giving this response something of the character of a T-dependent response, even though it is directed against a T-dependent/T-independent conjugate. However, this “trick’ is imperfect—although T-cells do assist, their assistance against conjugates is not as effective as it is against true T-dependent antigens. As a result, generally only low levels of antibody titers are elicited, and only some subjects respond to initial immunizations. Thus, several immunizations are frequently required. This poses a serious obstacle because patients are not always willing, or able, to complete this entire process. Furthermore, the process itself sometimes takes so long that patients contract the disease in a virulent form before they have been properly vaccinated.

In another attempt to gain the advantages of T-dependent response with T-independent antigens, including carbohydrates, researchers have attempted to discover T-dependent antigens which are structurally related to the T-independent antigen of interest. In theory, these structural mimics might elicit a superior immune response, compared to a vaccine based on either the original T-independent antigen alone or as part of a conjugate. Under this approach, at least, no part of the antigen in the vaccine is incompatible with T-cell assistance.

Yet locating T-dependent antigens which are sufficiently structurally related to T-independent antigens to be true immunological mimics has proven difficult. Researchers have taken three different approaches to this problem, each of which has serious limitations.

First, some researchers have succeeded in designing synthetic peptides which are immunologically mimetic to construct a protein structure which closely resembles the structure of the T-independent antigen of interest [209]. However, this approach is only as good as the researcher's knowledge of the various structures involved, which is frequently far from complete. Furthermore, a very high level of precision is required-higher because even a single amino acid error can have a profound effect on the immunogenicity of the synthetic peptide.

Second, some researchers have generated immunologic mimics by isolating anti-idiotypic antibodies which can elicit an immune response to carbohydrate antigens of S. pneumonia, P. aeruginosa, E. coli and Group A Streptococci [135, 172, 90, 131]. Anti-idiotypic antibodies are known to be structurally similar to the antigens of interest because of their design: they are generated against the idiotypes of antibodies which are known to specifically bind the carbohydrate of interest. As a result, the anti-idiotypic antibody and the carbohydrate bind specifically to the same idiotype structure (an antigenic determining structure in the antigen-binding portion of the carbohydrate binding antibody). Thus, much as two keys which fit the same lock have a high level of structural similarity, anti-idiotypic antibodies are thought to be structurally similar to the antibody-binding structures on carbohydrates. However, the similarity is not complete: these are still antibodies, isolated from the cells of mice, not complete carbohydrate structural mimics. As a result, there has been some concern that, for treatment of humans, human vaccines based on anti-idiotypic antibodies would be undesirable because of serious allergic reactions which could result [209]. This concern has led at least some researchers to seek alternative means of discovering T-dependent antigens which are structurally similar to T-independent antigens [209]. Finally, some researchers have sought to discover T-dependent antigens which are structurally similar to T-independent antigens by screening libraries of phages, which express hundreds of millions of random peptide sequences, using known carbohydrate-binding antibodies to find particularly promising peptides [72, 198, 75, 146]. The approach outlined in these references is sound only if one accepts that antigenic mimicry (meaning that the peptide mimic binds the same highly specific antibody as the carbohydrate of interest) is reasonably predictive of immunologic mimicry (meaning that the peptide will generate an immune response against the carbohydrate of interest). After all, if antigenic mimics are only rarely immunologic mimics, this procedure leaves one with far more peptide sequence candidates for immunologic testing after the antigenic screening step than can reasonably be tested. Indeed, after several failed attempts at obtaining an immunologic mimic using this approach were conducted, many in the art have in essence concluded that this approach is fundamentally flawed. In particular, at least one researcher has concluded that antigenic mimicry is rarely predictive of true immunologic mimicry, because the mechanism of peptide-antibody binding is different than carbohydrate-antibody binding [72].

Another serious limitation of both this approach and the design-approach of Westernik is that there is no a priori reason to believe that a peptide-based structural mimic necessarily exists for any given carbohydrate. The molecular basis underlying mimicry is unknown, and as such, offers no assurance that all carbohydrates structures have peptide mimics. There is certainly evidence in nature that some carbohydrate structures possess protein mimics. For example, the protein tendamistat is known to bind to the enzyme alpha-amylase at the same location this enzyme binds carbohydrates. And further research with synthetic peptides has demonstrated a certain level of mimicry in a variety of carbohydrates drawn from a number of species, although the theoretic basis for much of this data has been questioned [72]. Nevertheless, from these studies, it appears that each new carbohydrate presents a unique challenge to this area of research. Partly as a result of all of these limitations, there remains a need in the art for vaccines effective at the early stage of disease including but not limited to T-independent antigens and a method for developing such vaccines.

During the past decade the number of research on glycan/carbohydrates has rapidly increased to several hundred members and have been implicated in a variety of processes including cellular differentiation during development, a wide variety of physiological responses, regulating leukocyte trafficking and adhesion, cell-cell and cell-matrix interactions and immune regulation [116, 127, 176, 37, 39, 205]. Despite the fact that numerous human proteins with lectin-like activity have been identified few have been assigned biological functions. Most of the evidence for the involvement of these molecules comes from animal model studies or human hereditary diseases. These studies unfortunately provide only hints as to the possible mechanism of action, while the model organism are to simple to be of any use. The glycoconjugates described herein could be useful in bridging this gap. Glycosylation and glycobiology have been studied in a number of model organisms including yeast, dictostelium, C. elegans, sea urchins, drosophila and xenopus [108, 109, 2]. Each system has advantages and disadvantages. However all these simple organisms suffer from one major drawback, they do not display the complex genetics, development and behavior critical for understanding the role glycans play in higher organisms. As an alternative, researchers have constructed a variety of cell lines and transgenic mice including expressing glycosidases, masking glycosyltransferases, competing glycosyltransferases, and over expressing endogenous glycosyltransferases [64, 5, 74]. These studies indicate that glycans fulfill important physiological functions, however the systems are too complicated to delineate the mechanisms involved.

Glycans and glycoconjugates have been shown to have numerous biological activities e.g. cell adhesion, cell-cell interactions, pathogen-host interactions, toxins in cancer and inflammation processes, for example, carbohydrate binding proteins such as selectins are believed to play a critical role in immune responses including inflammation [177, 151] and as a modifier of the activity, stability and biological activity of proteins, and as immunogenic substances which have potential for vaccination against different diseases. An extensive literature has been developed during the last few years in this field [143, 56]. Specific carbohydrate ligands have been identified and have been used to control inflammation, immunosuppression (U.S. Pat. No. 5,576,305; U.S. Pat. No. 5,374,655). Other glycoproteins have also been shown to be useful in suppressing mammalian immune responses (U.S. Pat. No. 5,453,272).

Numerous human disease states are known to involve acquired (noninherited) changes in glycosylation and/or in the recognition of glycans, i.e. human glycophathologies. These alterations have been used as the basis for developing diagnostics, as therapeutics and/or as lead compounds for therapeutic vaccine/drug design. These include cardiovascular disorders, atherosclerosis, inflammatory skin diseases, diabetes mellitus, gastrointestinal infections, ulcerative colitis, hepatomas and liver disorders, cold agglutinin autoimmune disease, rheumatoid arthritis, nephrotic syndromes, neurological diseases, Alzheimer's disease, bronchial asthma, acute respiratory distress, shock, trauma and sepsis and cystis fibrosis [129, 12, 6, 54, 202, 99, 94, 214, 184, 86, 159, 130, 98, 149, 102, 25, 51, 83, 24, 66, 73, 129, 133, 87]. Glycoconjugates provide and methods describe herein maybe used as modulators of glycosylation and/or in the recognition of glycans in human glycophathologies.

Furthermore, altered glycosylation pattern is a feature common to all types of cancer cells and a number of glycan structures are well-studied markers for tumor progression [48, 32]. Many of the original “tumor-specific” antibodies were directed against carbohydrate epitopes. Glycosylation can be altered in many ways including altered branching of N-glycans, changes in the amount, linkage and acetylation of sialic acid residues, altered glycosaminoglycans, modified mucin glycosylation, altered Lewis structures, modified expression and shedding of glycosphingolipids, increased galectins and altered expression of blood group related structures [48, 32, 55, 183, 33, 217, 118, 18, 182, 145, 69, 27, 114]. Neoplasiainvolves alterations in gene expression that dramatically alter the growth, invasion and metastasizing capabilities of cells. The highly selective glycosylation changes seen in tumor cells that survive the selection process indicates that glycosylation plays a crucial role in these processes. Unfortunately, cellular heterogeneity in tumors and inherent genetic instability make it difficult to determine the functional consequences of specific glycosylation changes in such complex systems. Development inhibitors is one of step for personalize medicine cancer treatment. Current glycoconjugate technology has been unable to fulfill these demands.

Alteration in glycosylation patterns have also been shown during development and in numerous physiological responses including: fertilization, neurogenesis, organogenesis, hematopiesis and the immune system development [218, 49, 19, 23, 100, 101, 13]. The results from these and other studies show that glycans have a modulatory role in cell-cell interactions, cell-migration patterns during development and receptor activation responses.

Numerous pathogens use carbohydrate related interactions in order to gain entry into their hosts. For example, bacteria and intestinal parasites, such as amoeba, mediate the sugar specific adherence of the organisms to epithelial cells and thus facilitate infection [121]. Numerous parasites and other infectious diseases synthesize glycans binding proteins for attachment and invasion of host cells including: helminths, ascaris, hookworms, malaria, amoeba, intestinal and genital flaggellates [212, 170, 138]. Viruses such as influenza virus (myxovirus) and Sendia virus (paramyxovirus) use a haemagglutonin protein that binds sialic acid containing receptors on the surface of target cells to initiate the virus-cell interaction [150]. Furthermore, the differential expression of glycan binding molecules in organisms, for example on their cell surfaces are correlated with a pathogenicity and/or host specificity.

Bacteria produce a variety of glycoconjugates and glycans many of which are not present in eukaryotic organisms. The best known gram-negative bacterial glycolipid is Lipid A. Exposure to Lipid A results in fever and septic shock, and can result in death. The structure is known [156]. E. coli possess the O-antigen of which 170 variants have been identified [161]. Chlamydia trachomatis, the most common cause of blindness in the world today, makes a high-mannose-type N-glycan that is unique [141].

Other pathogenic microorganisms exploit host cell-surface glycoconjugates as receptors for cell attachment, tissue colonization and invasion [95]. These microbial adhesins present on the surface of bacteria, virus and parasites are involved in attachment to epithelial cells in the respiratory tract or the gastrointestinal tract and are required for infection [213, 174].

Delineating the targets would provide useful information for understanding the biology and developing therapeutic strategies [203].

Since most pathogens possess unique cell-surface carbohydrates, sequencing of oligosaccharides is a suitable research theme for upcoming carbohydrate-binding sites characterization studies. Specific carbohydrate structures are differentially expressed in each cell type are believed to be recognized by complementary molecule(s) expressed on the surface of the counterpart interacting cell. Complex carbohydrates directly involved in the recognition processes, including adhesion between cells, adhesion of cells to the extracellular matrix, and specific recognition of cells by one another [116, 127, 176, 37, 39, 205, 112, 140]. It is known that carbohydrate-carbohydrate interactions play an important role for the complimentary binding of glycosphingolipids and glycoconjugate-spore interactions [69, 27, 114, 120, 179].

Many natural bioactive molecules are glycoconjugates, and the attached glycans can have dramatic effects on the biosynthesis, stability, action and turnover of these molecules in the intact organism. Several classes of enzymes have been shown to be involved in the synthesis and degradation of glycans including: glycosidases that catalyze the hydrolysis of glycosidic bonds in a glycan, exoglycosidases that catalyze the cleavage of an internal glycosidic linkage in an oligosaccharide, endoglycosidases that catalyze the cleavage of a monosaccharide from the outer (nonreducing) end of a glycan or glycoconjugate, glycotransferases that catalyze the addition of monosaccharides and proteases that catalyze the cleavage of peptide bonds thereby altering the structural conformation of the glycoconjugate exposing different saccharides. Analysis of these enzyme pathways and their glycans and glycoconjugate products have been used to delineate numerous disease states, as diagnostic tools and in the development of therapies [103, 200, 137].

Glycoconjugate are attractive due to low immunogenicity, could stimulate T-independent and dependant immunity, longer shelf-life, may be administrated orally, intravenously, intraperitoneally, carbohydrates can be produced as homogeneous single compounds an a controlled manner with little or no batch-to batch variability, medicinal chemistry can modify synthetic carbohydrates to make more immunogenic, cheaper to produce synthetically [20, 96, 171, 3].

The preparation of synthetic glycoconjugates involves many protection and deprotection steps and that glycosylation creates a new stereocenter at the anomeric carbon [97]. Alternatively, enzymatic synthesis of oligosaccharides has shown promise in overcoming these drawbacks by eliminating the need of protecting groups and the “built-in” stereo specificity [148, 211]. Glycoconjugate is a moiety of a natural or chemically or enzymatically prepared carbohydrate molecule selected from the group consisting of monosaccharide, oligosaccharide, polysaccharide, glycolipid, glycoprotein, glycocompounds of any type with natural, modified natural or non-natural structure mimetics of carbohydrates and any of their combinations.

A vaccine is available for anthrax [84, 89]. Persons with high-risk occupations, such as laboratory workers and military forces, should receive the vaccine. In the case of suspected bioterrorism, ciprofloxacin or doxycycline should be given as chemoprophylaxis. The vaccine should be given concurrently, if available. The current vaccine licensed for use in the United States to protect humans against anthrax, anthrax vaccine adsorbed Biothrax (AVA Biothrax, also known as AVA or MDPH-PA) is prepared by adsorbing filtered culture supernatant fluids of the V770-NP1-R strain of a virulent strain of B. anthracis to aluminum hydroxide gel (Alhydrogel) [125]. In the United Kingdom, the human-use anthrax vaccine, alum-precipitated antigen (APA), is prepared by adsorbing filtered culture supernatant fluids of the 34F2 Sterne strain of B. anthracis to potassium aluminum sulfate (alum) [97].

PA of anthrax toxin appears to be the central component of all anthrax vaccines [157]. The PA-specific response to UK licensed anthrax vaccine was found to peak two weeks post immunization and fall back to pre-boost levels by week 12 [8]. Not surprisingly the wide range of results seen from the Swedish study suggests that the heterogeneity of the human host plays a significant role in modulating the extent of the PA-specific antibody response.

In fact, numerous studies have shown that both monoclonal and polyclonal antibodies to PA neutralize the anthrax toxin and function to provide immunity against the pathogen in animal studies [157, 60, 186, 124]. A recent report suggests that there are a number of limitations with the current, licensed vaccine for anthrax [157, 153]. It has also been suggested by this same group as well as others, that a well-characterized, safe, and effective vaccine is needed that provides immunity against anthrax [157, 186, 71].

Although the safety of both vaccines has been established, there are concerns over side effects of vaccination, despite evidence that observed adverse reaction rates for the US anthrax vaccine are comparable with those for other adult vaccines [85]. In some cases this has led to refusal of the vaccine by armed forces personnel [189]. Two surveys conducted by the American Department of Defense revealed that anthrax vaccination affected the work performance in a considerable proportion of vaccines, with up to 7.9% seeking medical advice and/or taking time off work [4]. These side effects may be caused by residual enzymatic components in the filtrate, which combine with PA to form active toxin complexes [186]. Some recipients developed hypersensitivity to unidentified components of the vaccine. It is possible that other factors in the vaccine including the aluminum adjuvant and preservatives may be responsible for adverse clinical outcomes. The current safety assessment of anthrax vaccine includes a skin test in rabbits to detect efficacy test (ET) and a weight gain test in mice, which receive one human vaccine dose intraperitoneally. Although the presence of ET can be ascertained by the skin test, the mouse test can not determine levels of residual toxin that do not cause weight loss or death, nor does this test identify specific toxins. In addition, the role other antigens may play in the toxicity of the vaccine remains undetermined.

While an anthrax vaccine is available [89] and can be used for the prevention of classic anthrax, genetic mixing of different strains of the organism can render the vaccine ineffective [142]. Compositions and methods should preferably not have the undesirable properties of promoting microbial resistance. Clearly, antipathogenic vaccine and immunoadjuvant that decrease the infectivity, morbidity, and mortality associated with sporeforming pathogens are needed. Furthermore, vaccine and immunoadjuvant that helped recognize and inhibited sporeforming pathogens preferable at the early state of infection/conditions prior symptoms/syndromes development caused by toxins and/or other products of pathogens are needed.

Therefore, a heretofore unaddressed need exists in the art to address the aforementioned deficiencies and inadequacies including a need to develop new inhibitor(s) that can rapidly and effectively destroy the B. anthracis or other sporeforming agents.

### SUMMARY OF THE INVENTION

The present invention provides glycoconjugates as inhibitors of spores of bacteria and others sporeforming agents that can cause disease/infection in living subjects such as human, animal, and plants, contamination of goods for consumption, food and beverages, and any other potentially infectable objects or articles.

The present invention, in one aspect, relates to a method of inhibiting spores of Bacteria genus. In one embodiment, the method comprises the step of administrating to a living subject in need thereof a pharmaceutical composition, wherein the pharmaceutical composition comprises at least one glycoconjugate having at least one disaccharide moiety or higher polysaccharide, and a pharmaceutically acceptable carrier. The glycoconjugate is a molecule containing a carbohydrate moiety that is glycosylated. The glycosylated carbohydrate moiety may comprise a glycoprotein or a glycolipid. The pharmaceutically acceptable carrier comprises at least one of a polymer, a glycoconjugate, water, oils, saline, a glycerol solution and any mixture thereof. The Bacteria genus includes B. cereus, B. thuringiensis, B. pumilus, and B. subtilis.

In another aspect, the present invention relates to a pharmaceutical composition, wherein the pharmaceutical composition comprises at least one glycoconjugate having at least one disaccharide moiety or higher polysaccharide, and a pharmaceutically acceptable carrier. The glycoconjugate is a molecule containing a carbohydrate moiety that is glycosylated. The glycosylated carbohydrate moiety may comprise a glycoprotein or a glycolipid. The pharmaceutically acceptable carrier comprises at least one of a polymer, a glycoconjugate, water, oils, saline, a glycerol solution and any mixture thereof. The Bacteria genus includes B. cereus, B. thuringiensis, B. pumilus, and B. subtilis. In yet another aspect, the present invention relates to an inhibitor for inhibiting spores of Bacteria genus, wherein the inhibitor comprises at least one glycoconjugate having at least one disaccharide moiety or higher polysaccharide. The glycoconjugate is a molecule containing a carbohydrate moiety that is glycosylated. The glycosylated carbohydrate moiety may comprise a glycoprotein or a glycolipid.

In a further aspect, the present invention relates to a method for inhibiting sporeforming pathogens or agents.

In one embodiment, the method comprises the step of administrating to a living subject in need thereof a pharmaceutical composition, wherein the pharmaceutical composition comprises at least one glycoconjugate having at least one disaccharide moiety or higher polysaccharide, and a pharmaceutically acceptable carrier. The glycoconjugate is a molecule containing a carbohydrate moiety that is glycosylated. The glycosylated carbohydrate moiety may comprise a glycoprotein or a glycolipid. The pharmaceutically acceptable carrier comprises at least one of a polymer, a glycoconjugate, water, oils, saline, a glycerol solution and any mixture thereof.

In yet another aspect, the present invention relates to a pharmaceutical composition, wherein the pharmaceutical composition comprises at least one glycoconjugate having at least one disaccharide moiety or higher polysaccharide, and a pharmaceutically acceptable carrier. The glycoconjugate is a molecule containing a carbohydrate moiety that is glycosylated. The glycosylated carbohydrate moiety may comprise a glycoprotein or a glycolipid. The pharmaceutically acceptable carrier comprises at least one of a polymer, a glycoconjugate, water, oils, saline, a glycerol solution and any mixture thereof.

In yet a further aspect, the present invention relates to an inhibitor of sporeforming pathogens or agents, wherein the inhibitor comprises at least one glycoconjugate having at least one disaccharide moiety or higher polysaccharide. The glycoconjugate is a molecule containing a carbohydrate moiety that is glycosylated. The glycosylated carbohydrate moiety may comprise a glycoprotein or a glycolipid.

In another aspect, the present invention relates to a method for inhibiting sporeforming pathogens or agents. In one embodiment, the method comprises the steps of binding the sporeforming pathogens or agents with at least one glycoconjugate and neutralizing the sporeforming pathogens or agents by identifying the binding glycoconjugate. The glycoconjugate is a molecule containing a carbohydrate moiety that is glycosylated. The glycosylated carbohydrate moiety may comprise a glycoprotein or a glycolipid. In one embodiment, the glycosylated carbohydrate moiety comprises at least one disaccharide moiety or higher polysaccharide. In another embodiment, the glycosylated carbohydrate moiety comprises a plurality of copies of same monosaccharide units or different monosaccharide units.

In yet a further aspect, the present invention relates to an inhibitor of sporeforming pathogens or agents, wherein the inhibitor comprises at least one glycoconjugate that is bondable to a sporeforming pathogen or agent. The glycoconjugate is a molecule containing a carbohydrate moiety that is glycosylated. The glycosylated carbohydrate moiety may comprise a glycoprotein or a glycolipid. In one embodiment, the glycosylated carbohydrate moiety comprises at least one disaccharide moiety or higher polysaccharide. In another embodiment, the glycosylated carbohydrate moiety comprises a plurality of copies of same monosaccharide units or different monosaccharide units.

A living subject can be a person, an animal, a plant, a tissue, one or more cells, or the like.

These and other aspects of the present invention will become apparent from the following description of the preferred embodiment taken in conjunction with the following drawings, although variations and modifications therein may be affected without departing from the spirit and scope of the novel concepts of the disclosure.

### DETAILED DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

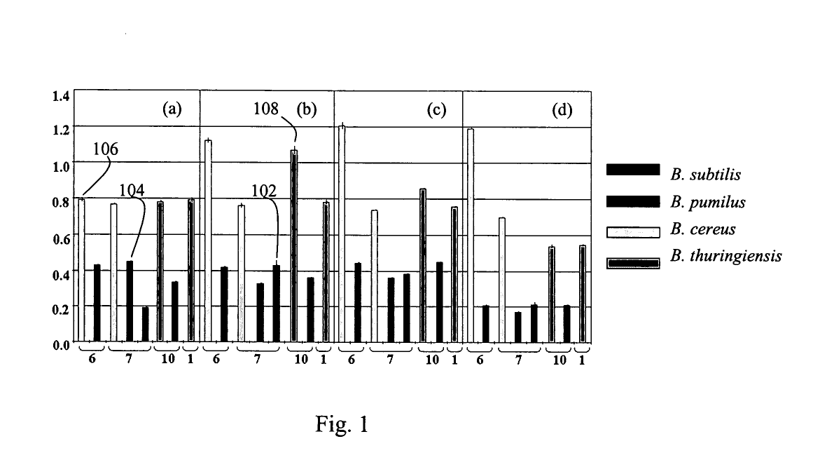

FIG. 1 shows a bar graph of binding of Bacillus subtilis, Bacillus pumilus, Bacillus cereus, Bacillus thuringiensis spores to glycoconjugates, respectively, using glycoconjugate solutions with different concentrations: (a) no dilution, (b) diluted at 10 times, (c) diluted at 100 times, and (d) diluted at 1000 times.

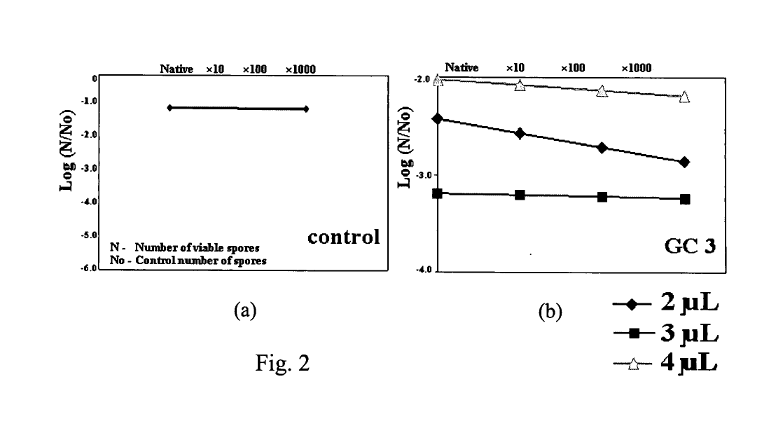

FIG. 2 shows inhibition curves of Bacillus subtilis: (a) a control, and (b) with 2 μl, 3 μl, and 4 μl of glycoconjugate No. 3, respectively.

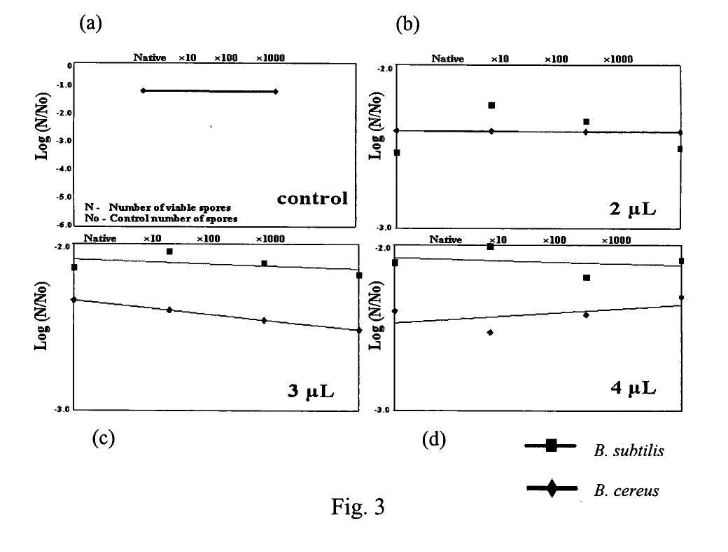

FIG. 3 shows inhibition curves of Bacillus subtilis vs. Bacillus cereus: (a) a control, (b) with 2 μl of glycoconjugate No. 3, (c) with 3 μl of glycoconjugate No. 3, and (d) with 4 μl of glycoconjugate No. 3.

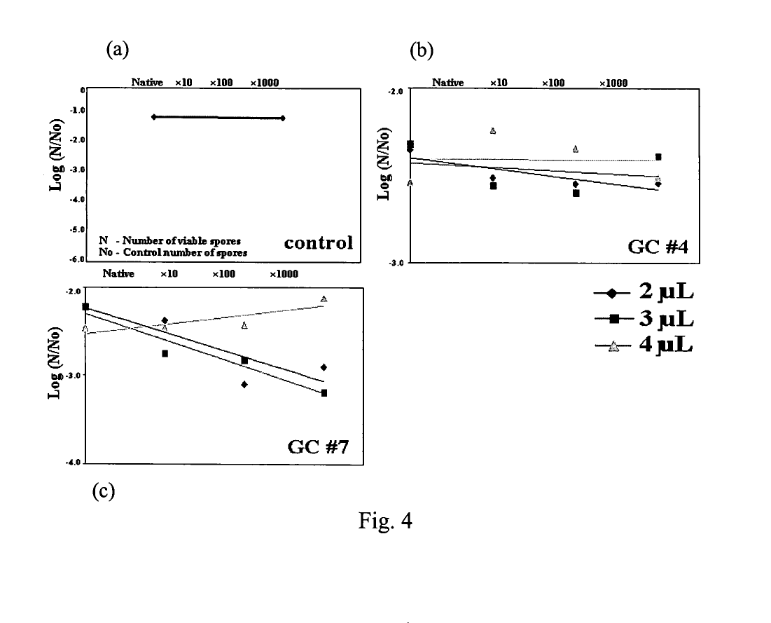

FIG. 4 shows inhibition curves of Bacillus cereus: (a) a control, (b) with 2 μl, 3 μl, and 4 μl of glycoconjugate No. 4, respectively, and (c) with 2 μl, 3 μl, and 4 μl of glycoconjugate No. 7, respectively.

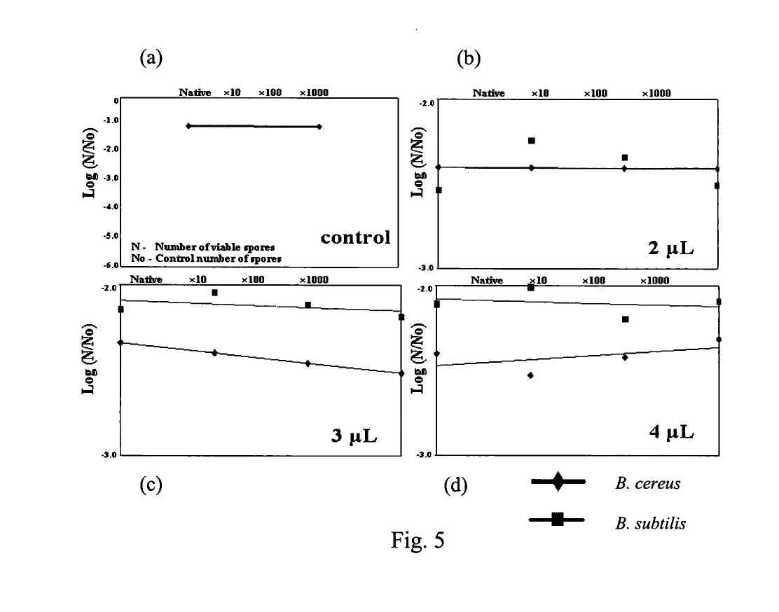

FIG. 5 shows inhibition curves of Bacillus cereus vs. Bacillus subtilis: (a) a control, (b) with 2 μl of glycoconjugate No. 4, (c) with 3 μl of glycoconjugate No. 4, and (d) with 4 μl of glycoconjugate No. 4.

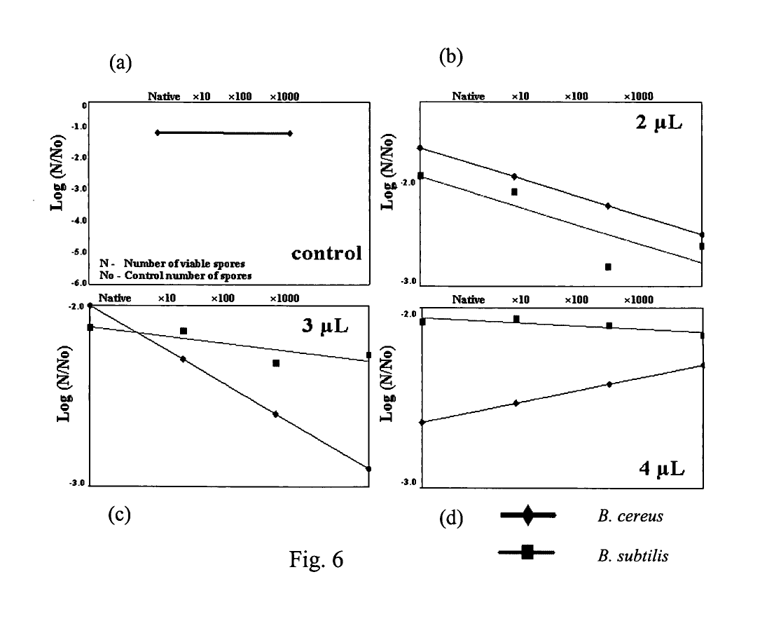

FIG. 6 shows inhibition curves of Bacillus cereus vs. Bacillus subtilis: (a) a control, (b) with 2 μl of glycoconjugate No. 7, (c) with 3 μl of glycoconjugate No. 7, and (d) with 4 μl of glycoconjugate No. 7.

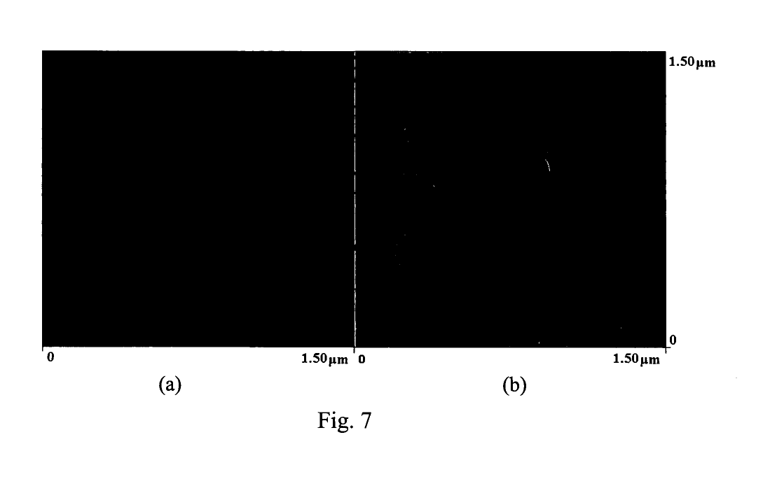

FIG. 7 shows AFM images of Bacillus cereus spores inhibition using glycoconjugate: (a) height, and (b) amplitude.

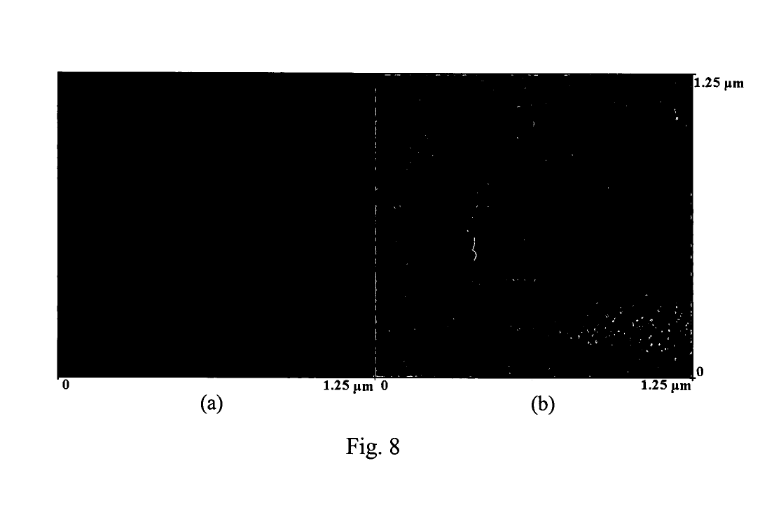

FIG. 8 shows AFM images of Bacillus subtilis spores inhibition using glycoconjugate: (a) height, and (b) amplitude.

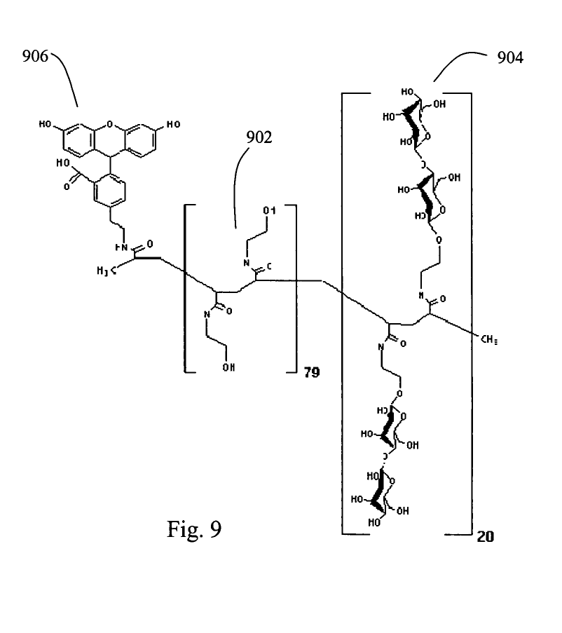

FIG. 9 shows a formula of disaccharide glycoconjugate bonded to polymer and fluorescein.

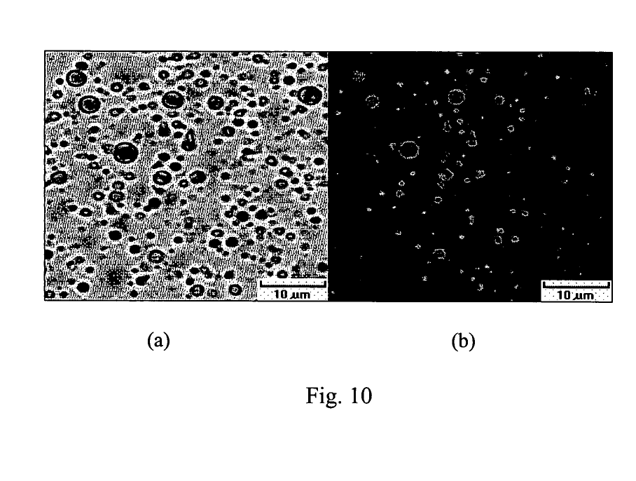

FIG. 10 shows images of glycoconjugate: (a) an optical microscope image, and (b) a fluorescence microscope image.

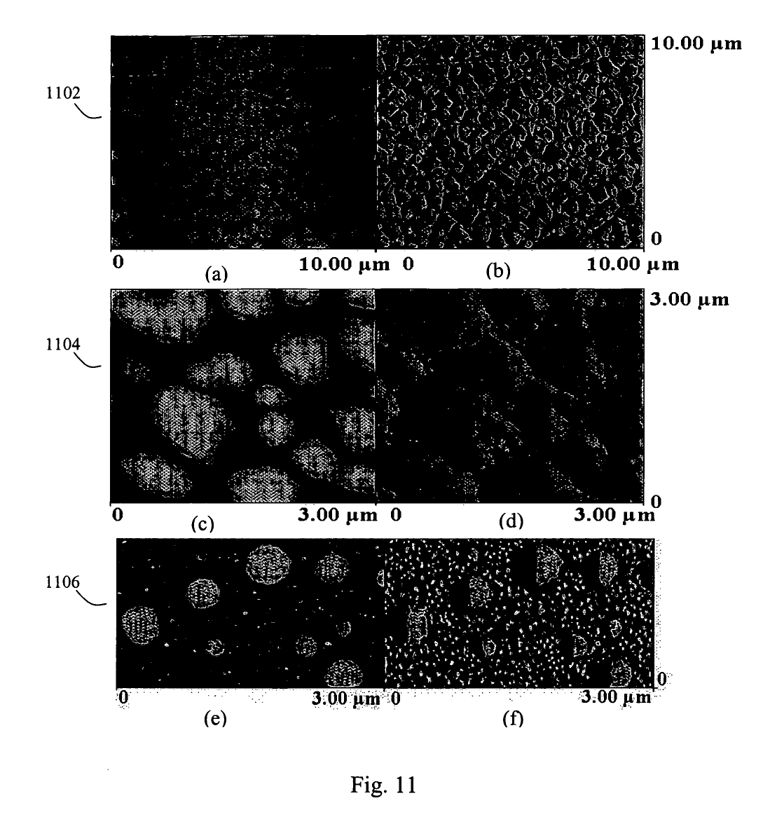

FIG. 11 shows AFM images of glycoconjugates: (a) height, with a low magnification for native glycoconjugates, (b) amplitude, with a low magnification for native glycoconjugates, (c) height, with a high magnification for native glycoconjugates, (d) amplitude, with a high magnification for native glycoconjugates, (e) height, with a low magnification for diluted glycoconjugates, and (f) amplitude, with a low magnification for diluted glycoconjugates.

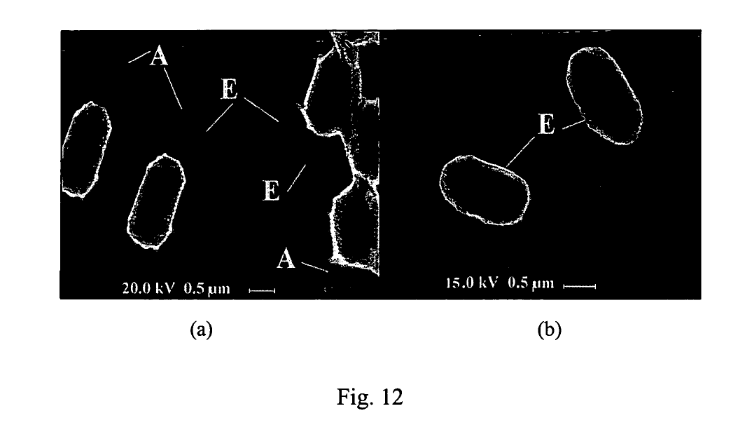

FIG. 12 shows SEM images of Bacillus cereus spores (a), and Bacillus subtilis (b), respectively, where A stands for appendages and E for exosporium.

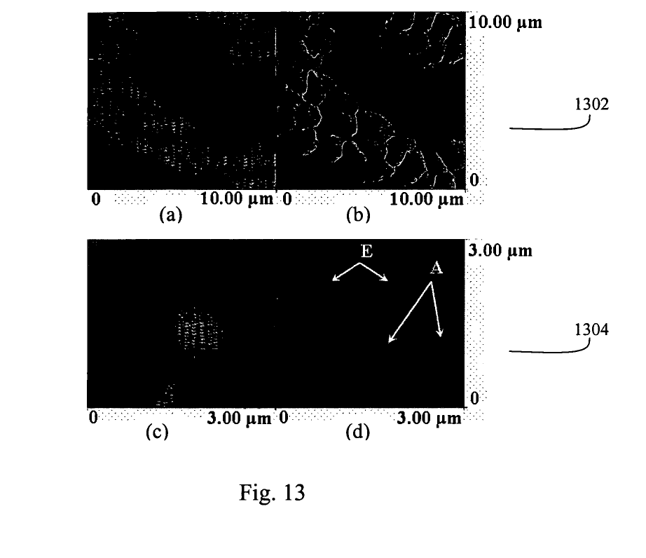

FIG. 13 shows AFM images of Bacillus cereus: (a) height, with a low magnification, (b) amplitude, with a low magnification, (c) height, with a high magnification, (b) amplitude, with a high magnification, where A stands for appendages and E for exosporium.

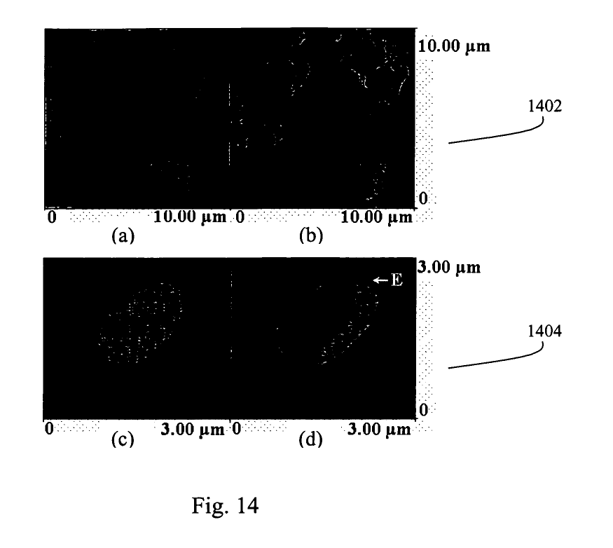

FIG. 14 shows AFM image of Bacillus subtilis: (a) height, with a low magnification, (b) amplitude, with a low magnification, (c) height, with a high magnification, (d) amplitude, with a high magnification.

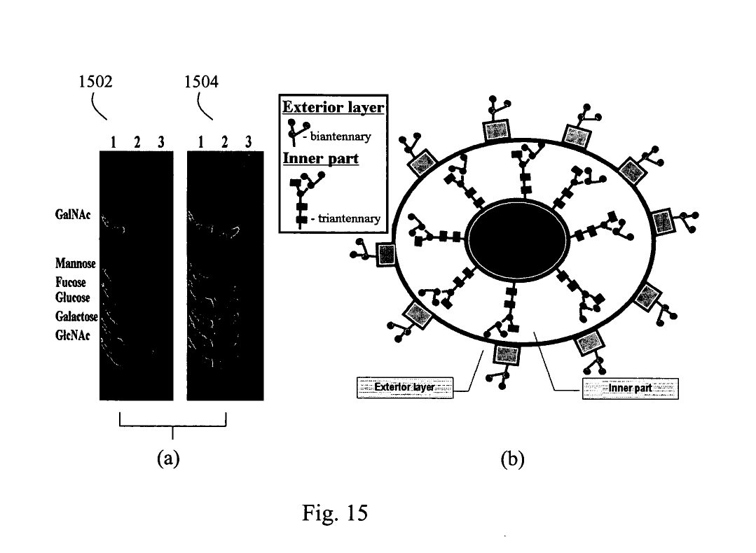

FIG. 15 shows (a) electrophoresis gel images of glycans of Bacillus cereus spores, and (b) a schematically structural diagram of glycans of Bacillus cereus spores.

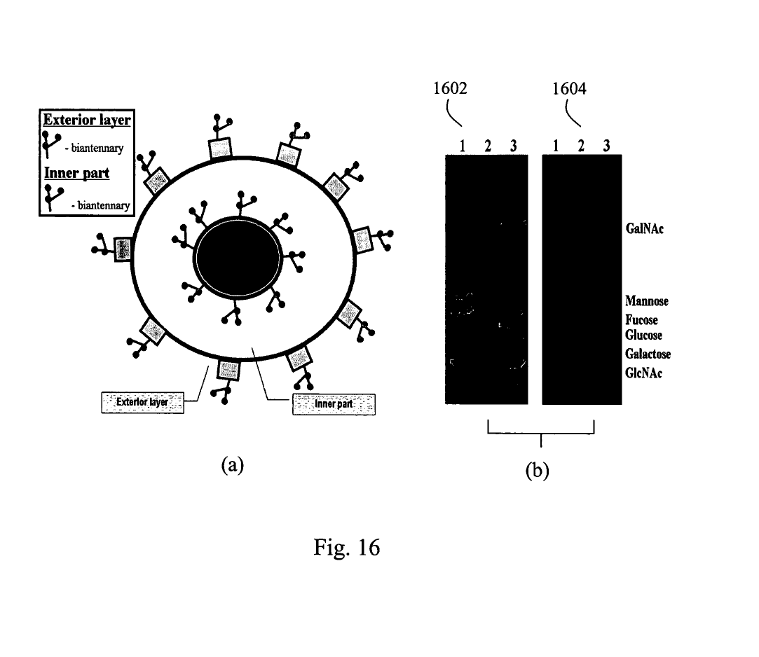

FIG. 16 shows (a) a schematically structural diagram of glycans of Bacillus subtilis spores, and (b) electrophoresis gel images of glycans of Bacillus subtilis spores.

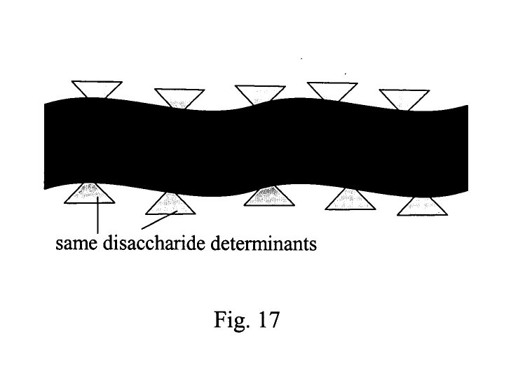

FIG. 17 shows schematically a diagram of glycoconjugate with same disaccharide or higher determinants.

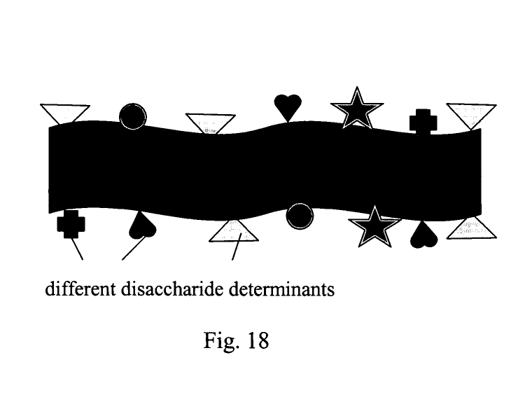

FIG. 18 is schematically a diagram of glycoconjugate with different disaccharide or higher determinants.

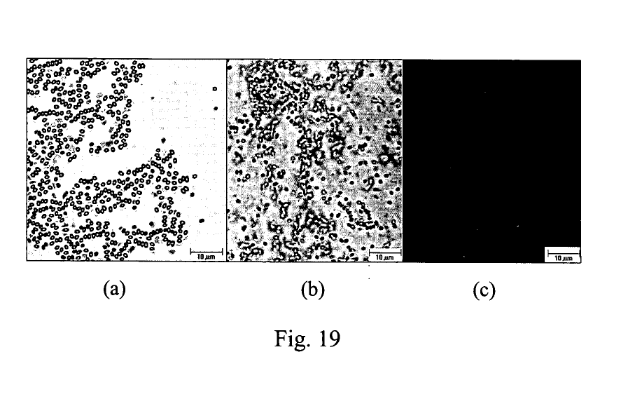

FIG. 19 shows (a) an optical microscope image of untreated spores, (b) an optical microscope image of spores binding to glycoconjugates, and (c) a fluorescence microscope image of spores binding to glycoconjugates.

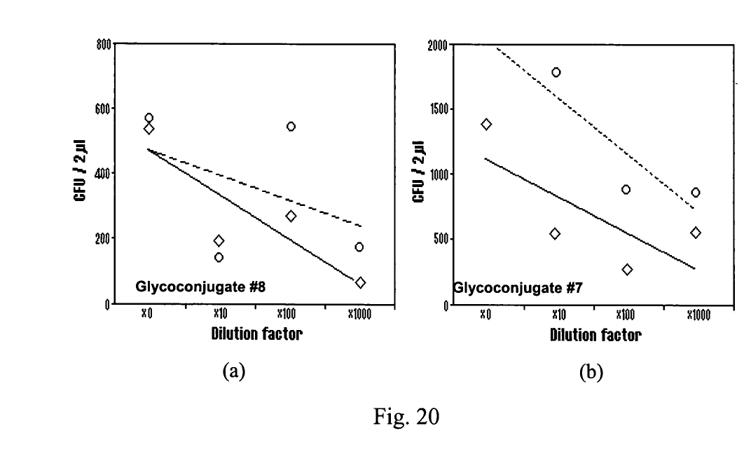

FIG. 20 shows relationship between dilutions and inhibition rate of Bacillus cereus and Bacillus subtilis spores based on CFU counts (a) using glycoconjugates No. 7 at different dilutions, and (b) using glycoconjugate No. 8 at different dilutions.

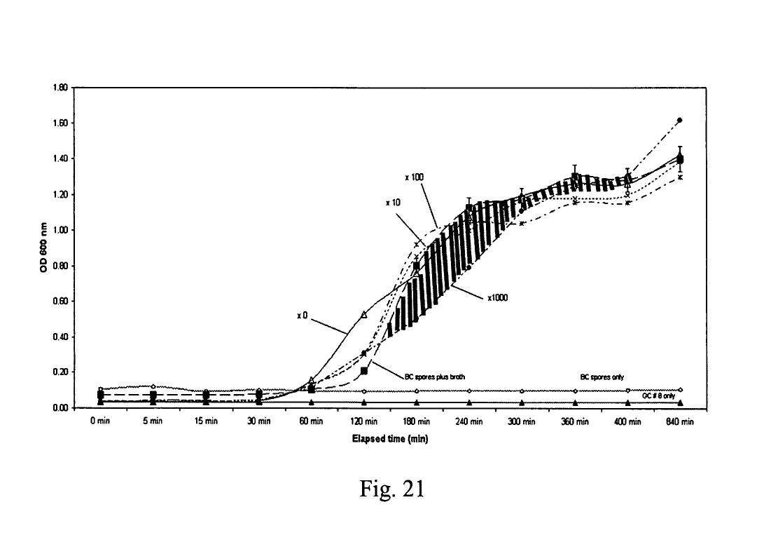

FIG. 21 shows glycoconjugates controlling germination of spores based on kinetics studies, where glycoconjugate is diluted at different times and untreated spore plus broth is used as a control.

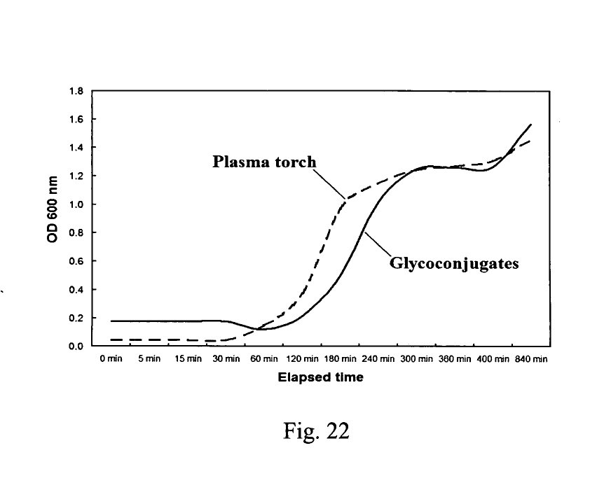

FIG. 22 shows a comparison of kinetics studies: inhibition of Bacillus cereus spores using glycoconjugates vs. plasma torch.

FIG. 23 shows schematically a chemical structure of agar.

### DETAILED DESCRIPTION

The present invention is more particularly described in the following examples that are intended as illustrative only since numerous modifications and variations therein will be apparent to those skilled in the art. Various embodiments of the invention are now described in detail. Referring to the drawings FIGS. 1-23, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “inhibiting” refers to inhibiting the activity of a entity such as sporeforming pathogen which is associated with the development of a particular disease state or medical condition and/or other conditions related to. The sporeforming agents can be inhibited by any mechanism, including, but not limited to binding of the spores by the polymer.

As used herein, in particular, the term “inhibition” refers to process of any manner of preventing, slowing, stopping of pathogen from any adverse reaction on human/animal/plant. In this invention, this refers to the ability of a given discrete glycoconjugate(s) to bind and, when present at an effective inhibitory level, to inhibit spore(s) or its component(s). Biologically relevant binding and inhibition event(s) can occur in a variety of modes. Spores may bind directly to the glycoconjugate moiety. In cases where the glycoconjugate expressed on spore itself is the target of the binding event. Glycoconjugates as inhibitors may act by altering the conformation, stability and the like, of the non-glycan moiety, hence altering the binding repertoire of said non-glycan moiety. Furthermore, additional component(s) may be required in order to develop the biologically relevant molecular recognition and inhibition repertoire of a given glycoconjugate or conjugates. A combination of any of these schemes is also possible. Effective inhibition level will be determined by usual experimental methods.

As used herein, an “effective amount” is an amount sufficient to inhibit or prevent, partially or totally, tissue damage or other symptoms/syndromes associated with the action of the spores and/or sporeforming pathogens within or on the body of the patient or to prevent or reduce the further progression of such symptoms/syndromes.

As used herein, the term “monomer”, as used herein, refers to both a molecule comprising one or more polymerizable functional groups prior to polymerization, and a repeat unit of a polymer. A copolymer is said to characterized by the presence of two or more different monomers.

As used herein, the term “polymer backbone” or “backbone” refers to that portion of the polymer which is a continuous chain comprising the bonds which are formed between monomers upon polymerization. The composition of the polymer backbone can be described in terms of the identity of the monomers from which it is formed, without regard to the composition of branches, or side chains, off of the polymer backbone. Thus, poly(acrylic acid) is said to have a poly(ethylene) backbone which is substituted with carboxylic acid (—C(O)OH) groups as side chains.

As used herein, the term “binding”, used with respect to a species of sporeforming pathogens, refers to a condition in which the species is attached to a conjugate(s) with an attractive force stronger than attractive forces that are present in the intended environment of use of the surface, and that act on the species. Binding may, e.g., mean covalent interaction, ionic interaction, hydrogen bonds, hydrophobic interactions and the like [204].

As used herein, the term “biomolecule” refers to all molecules of biological origin, be they natural (i.e. bacterial/fungi sporeforming pathogens) or engineered. This also includes synthetic or artificial molecules that have biological like characteristics using nanotechnology, biotechnology and the like.

As used herein, the term “target” in this context refers to a agent/pathogen including but not limited to bacterial and fungi spores, which is the object of attempted binding, inhibition and inactivation. The present invention also relates the of any sporeforming agents and their products, decontaminating goods for consumption, food and beverages, plants, buildings, equipments surfaces and any other potentially infected objects or articles colonized or otherwise infected by sporeforming agents and microorganisms. Terms “sporeforming agents” refers spores including pathogens infectious to humans, pathogens infectious to animals, pathogens infectious to herbs/plants/trees, pathogens occur in water, soil, food, beverages and any other objects and/or articles that can be infected and/or colonized.

As used herein, the term “glycan” refers to any saccharide or oligosaccharide, in free form or attached to another molecule and that can be hydrolyzed into these units.

As used herein, the term glycoconjugate may be used for any molecule which contains a carbohydrate moiety (which is glycosylated), like glycoproteins or glycolipids. “Glycoconjugate” also refers to any natural or non-natural non-carbohydrate compounds that mimic the physical, structural and chemical characteristics including the biological activities of natural or engineered functions of saccharides, and any combinations thereof. The term “glycoconjugate” also refers to a molecule comprised of a glycan moiety and possibly a non-glycan moiety, such that the interaction between the glycan and non-glycan moiety is sufficiently strong so as not to be released during the binding or inhibition unless this is done by design. In this context the term “non-glycan” moiety refers to any molecule of natural and non-natural origin including: naturally occurring macromolecules such as: proteins, lipids, RNA, DNA, biopolymers and the non-natural macromolecules with biological-like feature(s) produced using man-made engineering and fabrication design principles such as nano-injection molding or stamping, molecular imprinting or templating, as well as sequential-reaction based nanomaterials such as dendrimers, molecular rosettes, star-fish and the like.

As used herein, the terms “glycan” and “glycoconjugate” are used interchangeably herein. In this context, an “oligosaccharide” is defined as a branched or linear chain of monosaccharides attached to one another via glycosidic linkage [204].

As used herein, the term “monosaccharide” is defined herein as a carbohydrate that can not be hydrolyzed into a simpler unit. A monosaccharide can be of two types containing a carbonyl at the end of the carbon chain, termed an aldolase, or at the inner carbon, termed a ketose [204]. Common animal monosaccharides include for example:
Sialic acids: a family of nine-carbon acidic sugars the most common of which is N-acetyl neuraminic acid
Hexoses: six carbon neutral sugars, such as glucose, galactose and mannose.
Hexosamines: Hexose with an amino group at the 2-position, which can be either free or, more commonly, N-acetylated; N-acetylglucosamine, and N acetylgalactosamine.
Deoxyhexoses: six-carbon neutral sugars without the hydroxyl group at the 6 position, for example, fructose.
Pentoses: Five-carbon sugars such as xylose.
Uronic acids: Hexose with a negatively charged carboxylate at the 6-position such as glucuronic acid and iduronic acid.
   These monosaccharides dominate eukaryotic glycobiology but numerous variants can be found in lower organisms some of which are involved in numerous disease states [169, 91, 138].

As used herein, the term “glycome” refers to all glycans expressed in a cell and represents the next level of increasing biological complexity. This is due to the fact that the chemical structure of the monosaccharide bond allows two possible linkages and the formation of branched structures, as opposed to nucleotides and proteins which form only linear polymers and have only one basic type of linkage. As a result, the structural complexity of glycans is several orders of magnitude greater than proteins and DNA [122, 111].

As used herein, the term “binding” refers to any molecule that can undergo biological binding with a particular biological molecule. The term “binding region” refers to an area of a binding partner that recognizes a corresponding biological molecule and that facilitates biological binding with the molecule, and also refers to the corresponding region on the biological molecule. Binding regions are typified by nucleotides, molecular domains that promote van der Waals interactions, areas of corresponding molecules that interact physically as a molecular “lock and key”, and the like.

### OVERVIEW OF THE INVENTION

This invention in one aspect provides new wide-spectrum conjugate(s) that are suitable for the inhibition spores at early stage of invasion and/or infection in humans/animals and increase immunity. This invention also provides new wide-spectrum glycoconjugate(s) that are suitable for promoting microbial resistance in paint, coatings as well as other materials for buildings construction or electronics against sporeforming agents including bacterial spores and fungi.

In one aspect of the present invention, glycoconjugates are used as inhibitors of spores of bacteria and others sporeforming agents that may cause disease/infection in human/animal/plants, contamination of goods for consumption, food and beverages, and other potentially infectable objects. This invention can be also used to produce composites that exhibit enhanced resistance of composites in painting materials or buildings construction.

The present invention relates to inhibitors that are capable of decreasing the infectivity, morbidity, and rate of host mortality associated with a variety of spores and/or for the immunization as vaccine and/or immunomodulator and/or for treatment of infection even at the early stage. The present invention also relates to use of the novel conjugate derivatives and the like as an amplifier (potentiator) of plant defense responses induced by other materials (elicitors) in agriculture.

The present invention avoids the limitations of exciting vaccine against bacteria and/or their products. The present invention provides inhibitors of bacterial spores and others sporeforming agents that apply as vaccine and/or immunomodulators even at the early stage of infection/condition prior symptoms/syndromes development. The present invention also provides glycoconjugate(s) composites enhanced resistance for paint as well as for coating materials in buildings construction, agriculture and other industrial sectors. Furthermore, the present invention provides methods to analyze an inhibition efficacy.

Being primarily used as inhibition substrates, glycoconjugates played a major role in various embodiments this invention. Glycoconjugates facilitated the establishment of carbohydrate-carbohydrate and/or other interactions relevancy. Glycoconjugates located on the exterior surface of sporeforming pathogens serve as a potential multivalent receptor(s) display as shown in FIGS. 9 and 10, respectively. These glycoconjugates are recognized by other carbohydrates units of disaccharide and/or higher glycoconjugates, as shown in FIG. 11, leading binding. The technique presented in this invention can be helpful in finding, isolating prospective spore receptor recognition sites as shown in FIGS. 9 and 10, respectively, and discovering inhibitors essential for terminal neutralization of the spores.

As shown in FIGS. 4, 5 and 6, the inventors discovered that glycoconjugates have inhibiting capacities to sporeforming pathogens. The present invention demonstrated that disaccharide glycoconjugates were able to recognize and bind Bacillus spp. including B. cereus, B. thuringiensis, B. pumilus, and B. subtilis spores as shown in FIG. 11. Furthermore, the present invention demonstrated that diluted glycoconjugate solutions do not impede spore recognition as shown in FIG. 11, images 1102, 1104 and 1106. Moreover, glycoconjugates were observed to interact with receptor(s) epitopes expressed on the exterior layer of spores as shown in FIGS. 8 and 9. Taken together, a major advantage of the present invention lies in the glycoconjugate specificity and selectivity to spore species. Multiple aspects of recognition lead to applications like the recognition of specific sporeforming and/or other pathogens. The findings of the present invention provide new a opportunity on how to improve the binding ligands selection, indispensable for the development of inhibitors as shown in FIGS. 5 and 6. In addition, the findings provide new information for inhibition of bacterial spores and/or sporeforming pathogens.

Thus, the present invention relates to binding, as shown in FIG. 11, and inhibition of spores of Bacteria genus including B. cereus, as shown in FIGS. 18-23, and other sporeforming bacteria such as B. subtilis, as shown in FIGS. 13-17, which represent carbohydrate binding epitopes on exterior layers, which can be practiced by the steps of:
(1) binding of sporeforming agents by specific glycoconjugate with at least one disaccharides glycoconjugate; and
(2) inhibiting and terminal neutralization of sporeforming pathogens by identifying binding glycoconjugates.

Then, one can isolate and select one or more of the glycoconjugates or fragments to which the spores have bound and inhibited as highly effective moiety for vaccine and/or immunomodulator.

A glycoconjugate which recognize, inhibit and “immunologically mimic” an antibody is a substance will increase an immune response against sporeforming agents. The glycoconjugate is preferably with specific repeated or non-repeated at least one disaccharide glycoconjugate units of any length are within the scope of the invention.

The glycoconjugates of the present invention may protect a living subject at the early stage of disease by binding and inhibition and than protect against symptoms/syndromes of infection/condition or to ameliorate the effects of infection/condition caused by sporeforming agents. Any bactericidal assay that is known in the art may be used to identify the protective antibodies of the invention, although the assay set forth in U.S. Pat. No. 5,971,511, which is incorporated herein for background information only by reference in its entirety, is preferred. In addition, protective effect can be identified by use of the lethal challenge assay in which laboratory animals, generally mice, are injected with a lethal amount of the spores being tested. Glycoconjugates will be then administered and mouse survival will be determined. Those glycoconjugates that are able to protect against death are considered to be protective.

Once the glycoconjugates with high inhibition values are determined by binding and inhibiting screening processes, they may be used as vaccine or immunomodulator. The vaccine or immunomodulator may include the glycoconjugate itself or the glycoconjugate units may be conjugated to a carrier or otherwise compounded. Glycoconjugate preferably can activate and recruit T-cells and thereby augment T-cell dependent antibody production. However, the carrier such as a polymer need not be strongly immunogenic by itself and do not affect binding. Polymeric compounds which present ligands in multivalent form can lead, in ligand-receptor recognition processes, to increased interactions with receptors and be used therapeutically, for example as receptor blockers or multivalent enzyme inhibitors [115].

Other applications have also been reported for polymers which are functionalized by defined quantities of active molecules. For targeting active substances, use can be made of more complex polymers and/or carriers which, in addition to active compounds, also carry substances which are selectively recognized by particular cell-surface receptors so that these active compounds are preferentially transported to these types of cells [104].

In addition, active compounds which are coupled covalently to polymers by way of labile bonds can be selectively released in the organism at sites where these bonds are cleaved by particular physiological conditions [216, 215, 61, 62].

The molecular weight of the polymer as carrier is not critical, but is, preferably, suitable for the intended mode of administration and allows the polymer to reach and remain within the targeted region of the body. For example, a method for treating an intestinal infection may utilize a polymer of sufficiently high molecular weight or degree of cross-linking to resist absorption, partially or completely, from the gastrointestinal tract into other parts of the body. Preferably, if linear, the polymer to be administered has a molecular weight ranging from about greater than 1 to about 1 million Daltons or more, such as 2,000 Daltons to about 500,000 Daltons, 5,000 Daltons to about 150,000 Daltons, or about 25,000 Daltons to about 1 million Daltons. Alternatively, the molecular weight can be from about 100,000 to about 1 million or between about 400,000 to about 1 million Daltons. The polymers of use in one embodiment of the present invention are preferably substantially nonbiodegradable and nonabsorbable. That is, the polymers do not substantially break down under physiological conditions into fragments which are absorbable by body tissues. The polymers preferably have a nonhydrolyzable backbone, which is substantially inert under conditions encountered in the target region of the body. For instance, polystyrene sulfonate polymer might be use for delivery into the gastrointestinal tract. Preferably, the polymer is a soluble, uncrosslinked polystyrene sulfonate polymer having a molecular weight between about 400,000 and 1 million Daltons, such as 600,000 Daltons. Alternatively, polymer backbones which are suitable for the present invention include polyacrylamide, polyacrylate, poly(vinyl) and poly(ethyleneimine), polystyrene backbones as well. A co-polymer can comprise a combination of two or more of these backbone elements. The polymer to be administered can also be condensation polymer, such as a polyamide or a polyester [193].

The quantity of a given glycoconjugate polymer to be administered is determined on an individual basis and, at least in part, by consideration of the individual's size, the identity of the known or suspected pathogenic organism, the severity of symptoms to be treated and the result sought. The glycoconjugate polymer can be administered alone or in a pharmaceutical composition comprising the polymer and one or more pharmaceutically acceptable carriers, diluents or excipients.

Strong inhibitors specifically to different species of sporeforming agents are within the scope of this invention as well. Multiple copies of monosaccharide units are also within the scope of this invention as shown in FIG. 11. Multiple copies of monosaccharide units are also within the scope of the invention, either unconjugated or conjugated to one or more copies of the carrier. Different monosaccharide units of the glycoconjugate are also within the scope of the invention as shown in FIG. 11, either alone or in combination with each other, not necessarily identically reproduced, and either unconjugated or conjugated to one or more copies of the carrier. In a further embodiment, spores that express the carbohydrate unit binding epitopes are within the present invention.

In a preferred embodiment, the carrier is glycoconjugate with similar or distinct monosaccharide units that inhibit spores as vaccine/adjuvant or any other compound capable of enhancing the immune response.

The glycoconjugate(s) may be selected for a group having viral, bacterial, and fungal spores. In a more preferred embodiment, the carrier is polymer. The carrier can be other moieties to which a specific glycoconjugates bonded, all of which may be obtained from binding and inhibiting by methods known in the art. Glycoconjugates may function as carriers as well. The selected glycoconjugates with high inhibition values may be immunogenic or, alternatively, the immunogenicity may arise from the compounding at least one disaccharide glycoconjugate and/or higher to carrier(s). Immunogenicity will be measured by standard immunological methods. A particularly preferred means of determining the immunogenicity of a given substance is to first obtain sera of mice both before and after immunization with the glycoconjugate substance. Following this, the strength of the post-immunization sera is ascertained using an ELISA, and compared against the ELISA results obtained for the pre-immunization sera.

Glycoconjugates are attractive on the basis of low immunogenicity, ability to stimulate immunity, longer shelf-life, different way of delivery in organism, and availability of methods of synthesis and manufacturing [20, 96, 171, 3].

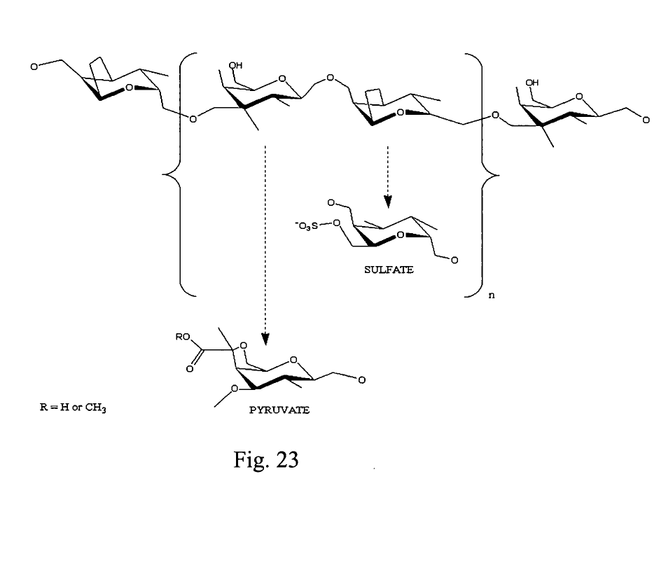

The glycoconjugates as vaccines/immunomodulators may enhance immunogenicity as a mode of action. Enhanced immunogenicity refers to immunogenicity greater than that obtained by present anthrax vaccines alone, and preferably that sufficient to affect a statistically measurable immunoprotective effect. As a point of reference, a glycoconjugate would certainly have enhanced immunogenicity if it provoked a level of immunogenic response equal to or greater than that obtained by administration of present anthrax vaccine. This invention may also benefit of stimulation of T-independent as shown in FIG. 27 and T-dependent as shown in FIG. 28 immunity, respectively. One aspect of the present invention is conversion glycoconjugates into optimally immunogenic molecules. Carbohydrates frequently function as T-independent antigens, which can not be properly processed by the antigen presenting cells that begin the typical mammalian immune response. By contrast, T-dependent antigens are initially processed by antigen presenting cells and then rely on T-cells to stimulate B cells to manufacture large quantities of antibodies against the antigen. As a result of these molecular biological differences, T-dependent antigens are immunologically superior to T-independent antigens, including carbohydrates, in three ways:
(1) T-dependent antigens are remembered by the immune system while T-independent antigens are not. Thus, after vaccination, an infection with a T-dependent antigen will be met with an extremely swift and concentrated antibody attack compared to the response to the initial vaccination. Infections with T-independent antigens, by contrast, generally receive the same level of antibody response, even after vaccination;
(2) T-dependent antigens are met with specific antibodies of increasing affinity against them over time, while T-independent antigens are met with antibodies of constant affinity; and
(3) T-dependent antigens stimulate a neonatal or immature immune system more effectively than T-independent antigens.

Pharmaceutically acceptable carriers can be used to practice the present invention include sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described earlier [160]. These carriers can also contain immunoadjuvants, including but not limited to alum, aluminum compounds (phosphate and hydroxide), and muramyl dipeptide derivatives.

The present invention also relates to the treatment of a patient by administration of an immunostimulatory amount of the vaccine and/or immunomodulators. Patient refers to any living subject for whom the treatment may be beneficial and includes mammals, especially humans, horses, cows, dogs, and cats as well as other animals, such as chicks. In an agricultural or horticultural application of the invention from inhibitory applications point of view, plants at risk from sporeforming pathogens may also be considered as “patients”. An immunostimulatory amount refers to that amount of vaccine that is able to stimulate the immune response of the patient for the prevention, amelioration, or treatment of symptoms/syndromes and/or diseases. Of course, as noted above, the immunostimulation may result from the form of the antibody or the adjuvant with which it is compounded.

Glycoconjugates of the present invention may be administered by any route, but are preferably administered topically, mucosally or orally. Other methods of administration will be familiar to those of ordinary skill in the art, including intravenous, intramuscular, intraperitoneal, intracorporeal, intrarticular, intrathecal, intravaginal, intranasal, oral and subcutaneous injections.

The present invention described here applies to the next level of complexity namely glycoconjugates for recognition and inhibition of any sporeforming agents, and/or for vaccine/immunomodulators development for early treatment any pathological symptoms/syndromes or any conditions caused by sporeforming agents.

In addition to this chemical complexity, a common feature of protein glycosylation is that at any given glycosylation site on a given protein, synthesized by a particular cell type, a range of variations can be found in the structure of the glycan. These minor glycosylation variants are referred to as microheterogeneities. Thus, a given glycoprotein can exist in numerous glycoforms, i.e. different molecular forms of a glycoprotein resulting from variable glycan structure and/or glycan attachment site occupancy. The origin of this microheterogeneity lies in the dynamics of the sequential nature of the glycosylation process. The glycoconjugates of the present invention make it possible to investigate the recognition, and inhibition of all possible types of glycans with their natural or non-natural ligands.

One advantage of the glycoconjugate according to the present invention is the ability to assay the same recognition, interaction, and inhibition in multiplicity under essentially identical conditions or other natural variations.

The involvement of oligosaccharides in cell-cell recognition by the immune system in response to inflammation [112], and sperm-cell recognition during fertilization [140] are but a few examples. It is also known that modifying the expression of glycosides and glycosyltransferases interferes with normal development. Glycoconjugates can be used also in the experiments with different cells/cells lines and their fragments including, but not limited to, microorganisms, bacteria and viruses containing the listed above glycan recognizing molecules and any other glycan binding sites and their inhibitors.

The present invention may be based on biological binding between the glycan/glycoconjugates or their derivatives and a molecule/substances such sporeforming pathogens and/or receptor(s) network by preventing, slowing and stopping life activity by inhibition and/or increasing resistance and or immunity. In this context the term “biological binding” refers to the interaction between a corresponding pair of molecules, i.e. binding partners, that exhibit mutual affinity or binding capacity, typically specific or non-specific binding or interaction, including biochemical, physiological, and/or pharmaceutical interactions. Biological binding defines a type of interaction, preferably by recognition regions, that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/bacteria/spore/fungi/ligand, etc.

With the glycoconjugates of the present invention it is now possible to bind, identify and inhibit such sporeforming pathogens including but not limited to bacterial and fungi spores described above. Accordingly, the present invention also relates to a method of inhibition species, wherein a signal pattern is generated that is unique to a particular organism, and the species is a biological sample from a particular organism and related standard is the pattern normally found in that organism.

Thus, the present invention also relates to the use of the glycoconjugate(s) for binding and inhibition of biological warfare agents and/or other sporeforming agents and/or as vaccine, immunomodulator and/or antimicrobial agents.

The majority of the studies cited above pertain to human. Study of other mammalians indicates that the majority of glycan alterations exist but often show species specific glycan alterations. Thus, a preferred embodiment of the glycoconjugate(s) would be for use in veterinary science for as therapeutic/immunomodulators agents, and the like.

Furthermore, the glycoconjugate(s) of the present invention can be used in the field of plant biology. In plants glycans are used extensively for structural integrity, i.e. the cell wall, as well for the purposes described above [173]. The cell wall is a source of small glycans that are used to signal patterning and morphogenesis, i.e. mammalian equivalent to hormones, as well as to signal pathogen invasion which activates a signaling cascade that turns on defense genes. Defensive oligosaccharides have between 4 and 20 saccharide residues [68]. Glycans also serve an important role in the symbiotic relationship between nitrogen-fixing bacteria and plants [119, 162]. Complex plant glycans have been shown to be highly immunogenic in mammals, with the potential for inducing allergic responses [52].

Thus, an additional preferred embodiment of the invention in the field of plant science is the enhancing resistance of plants to plant infections produced in plants including natural compounds and those introduced by natural plant breeding or of genetically engineered plants.

The use of the glycoconjugates and methods of the present invention can be extended to a wide range of applications that require complex sample decontamination to complex samples containing biological material and/or degradation products including but not limited to such as food stuffs like beverages, dry foods, and the like (including but not limited to quality control, for inhibition of unwanted microbial growth, freshness, physical damage), as well as the control of environmental samples for microbial flora (including but not limited to microbial content and composition), polluants and their breakdown products in air, soil and water samples. This strategy and assays based on it could also be used for monitoring fermentation processes, including but not limited to yogurt, beer, wine and the like, broths, as well as in fermentation processes in which products are produced such as biological compounds produced by microbial processes, such as insulin from genetically engineered bacteria and the like, as well as condiments made for seasoning and the like, as well fermentation processes used in the production of animal food stuffs.

Furthermore, the use of the glycoconjugates and methods of the present invention can be extended to a wide range of applications that require resistance against sporeforming agents.

### EXAMPLES AND IMPLEMENTATIONS

Without intent to limit the scope of the invention, further exemplary methods and their related results according to the embodiments of the present invention are given below. Note again that titles or subtitles may be used in the examples for convenience of a reader, which in no way should limit the scope of the invention. Moreover, certain theories are proposed and disclosed herein; however, in no way they, whether they are right or wrong, should limit the scope of the invention.

### Additional Experiments and Results

Methods

1.1. Materials

Disaccharide-PAA-flu glycoconjugates were purchased from GlycoTech Inc. (Rockville, Md.). Tween 20, 30% albumin bovine solution, culture tissue grade water: W3500, 2,2′-azino-bis(3-ethylbenzthiazoline-6-sulfonic acid) liquid substrate system, standard proteins were purchased from Sigma-Aldrich (St. Louis, Mo.). Goat anti-mouse (IgG+IgM)-horseradish peroxidase (HRP) conjugate was purchased from Roche (Indianapolis, Ind.). Gold seal® cover glasses were obtained from Fisher Scientific (Pittsburgh, Pa.). Tryptic Soy Agar (TSA) media was purchased from Raven Biological Laboratories Inc. (Omaha, Nebr., USA). Organ Tissue Culture dishes were purchased from Falcon Plastics (Oxnard, Calif., USA). Anhydrous calcium sulfate® was purchased from W.A. Hammond Drierite Company LTD. (Xenia, Ohio, USA). Ultra-dishes Petri were obtained from PGF Scientific (Gaithersburg, Md., USA). Sterile tips were purchased from VWR International (Bridgeport, N.J., USA).

1.2 Bacterial Spores

Sterile bacterial spore suspensions, namely B. cereus ATCC 11778 (3.5×10⁶ CFU/0.1 mL), B. thuringiensis ATCC 29730 (3.5×10⁶ CFU/0.1 mL), B. subtilis ATCC 9372 (3.5×10⁶ CFU/0.1 mL), and B. pumilus ATCC 27142 (3.5×10⁶ CFU/0.1 mL were purchased from Raven Biological Laboratories Inc. (Omaha, Nebr., USA).

2.1 Glycoconjugates

The fluoresceinated glycoconjugate (Glyc-PAA-flu) is comprised of a soluble polyacrylamide (PAA) backbone (˜30 kDA) with various carbohydrate side groups (20% mol) and fluorescein (flu), as shown in FIG. 9. PAA-flu is known to be an inert carrier due to the lack of charged groups and, therefore, does not show any specific binding to cells [21, 22, 57]. The suppleness of the PAA backbone facilitates the conjugates to adjust themselves to targets. Glyc-PAA-flu contains 1% mol flu (2-3 flu residues per polymer chain), sufficient for labeled detection techniques such as fluorescence microscopy, as shown in FIG. 10.

3.1 Study of Glycoconjugates-Spore Binding

In practicing the present invention, fluoresceinated glycoconjugates (GlycoTech Inc., Rockville, Md.), as shown in FIGS. 1, 2 and 3, were employed as synthetic ligands to bind and inhibit, as shown in FIGS. 9, 10 and 11, respectively, B. cereus, B. subtilis, B. pumilus, and B. thuringiensis spores (targets), which is also shown in FIG. 1. Solutions of disaccharide glycoconjugates were prepared according to the manufacture's protocol [63]. The solutions were then diluted ×10, ×100, ×1000 into a PBS/0.2% NaH₃ buffer. The binding studies were performed according procedure as described early [179]. Spore binding with glycoconjugates was confirmed by optical and fluorescent microscopy as shown in FIG. 19.

4.1.1 “Simulated Life Environment” Fabrication

The inventors have fabricated “simulated life environment”. In this context the term “simulated life environment” in particular refers as environment close to human/animal in terms of amino acids, proteins, carbohydrates, lipids, t ⁰C and pH present as a surface on a solid substrate. A surface may have steps, groves, trenches, ridges, terraces, and the like.

In particular, the term “solid support” refers to any material insoluble in a medium that used as support for “simulated life environment”. Preferably, the solid support is plastic, on which a self assembling layer(s) of “simulated life environment” can be coated. In that case, Tryptic Soya Agar (TSA) as media of “simulated life environment” is immobilized on solid support. Preferably, said solid support most preferably with media. In principle, the “simulated life environment” surface on which included life support nutritional factors can be produced according to methods well known to person skilled in the art, which also provides examples for appropriate chemical composition which can be employed in accordance with the present invention, if necessary in modified form. TSA conforms with the formula (Table 1). The cultivation of a wide variety of microorganisms is known in the art [113, 191, 158]. TSA used in environmental monitoring, in multiple water and wastewater applications, in numerous standard methods for food testing, culture collections, and testing bacterial contaminants in cosmetics [147, 59, 192, 14, 28]. Temperature 37° C. was controlled using Precision® Mechanical Convection Incubator Model 30M (Winchester, Va.). pH was 7.3 (Table 2).

TABLE 1
Tryptic Soya Agar (TSA) Formula/Liter

| | |
|---|---|
| Enzymatic Digest of Casein | 15 g |
| Enzymatic Digest of Soybean Meal | 5 g |
| Sodium Chloride | 5 g |
| Agar | 15 g |
| Final pH | 7.3 ± 0.2 at 25° C. |

Detailed chemical composition and description of enzymatic digests of casein, soybean meal agar as main compounds of 2-dimetional “simulated life environment” has shown at the Tables 3, 4, 5, and 6, respectively. Enzymatic digests of casein and soybean meal provide the proteins, nitrogen, vitamins and carbon. The natural sugars of soya promote bacterial growth. Sodium chloride maintains osmotic balance. Agar is the solidifying agent.

TABLE 2
Chemical composition and characteristics “simulated life environment”

| | |
|---|---|
| Protein (%) | 51.6 |
| Fiber (%) | 3.78 |
| Fat (%) | 5.58 |
| Urease Activity (%) | 0.04 |
| Protein Solubility (KOH) (%) | 86.24 |
| Threonine (%) | 1.87 |
| Cysteine (%) | 0.76 |
| Methionine (%) | 0.68 |
| Lysine (%) | 2.98 |
| Tryptophan (%) | 0.67 |
| Total Amino Acids (%) | 46.53 |
| Lactose (%) | 4.9 |
| Minerals (%) | 1.8 |
| Vitamins (%) | 0.2 |
| Total solids (%) | 15.0 |
| t ° C. | 37 |
| pH | 7.3 |

4.2.1 Soybean Meal

Soybean meal is the product remaining after extracting most of the oil from whole soybeans. The oil may be removed by solvent extraction or by an expeller process in which the beans are heated and squeezed [79].

TABLE 3
Composition of Soybean Meal

| | |
|---|---|
| Crude Protein (%) | 47.95 |
| Crude Fiber (%) | 3.78 |
| Crude Fat (%) | 1.48 |
| Urease Activity (%) | 0.04 |
| Protein Solubility (KOH) (%) | 86.24 |
| Threonine (%) | 1.87 |
| Cysteine (%) | 0.76 |
| Methionine (%) | 0.68 |
| Lysine (%) | 2.98 |
| Tryptophan (%) | 0.67 |
| Total Amino Acids (%) | 46.53 |
| Calcium (%) | 0.02 |
| Phosphorus (%) | 0.65 |

The higher quality soybean meals comes from the higher digestibility plus lower fiber levels and consistently high in protein, and amino acid levels. The lower fiber level meals will probably be reflected in higher energy values [11]. The results of the soybean meal analyses were as shown in Table 3 [11, 107, 45, 79].
4.2.2 Casein

The nitrogen content of milk [76, 199, 42, 117, 46] is distributed among caseins (76%), whey proteins (18%), and non-protein nitrogen (NPN) (6%). This nitrogen distribution can be determined by the Rowland fractionation method. The concentration of proteins in milk is as shown in Table 4:

TABLE 4
Milk Composition

| Milk | grams/liter | % of total protein |
|---|---|---|
| Total Protein | 33 | 100 |
| Total Caseins | 26 | 79.5 |
| alpha s1 | 10 | 30.6 |
| alpha s2 | 2.6 | 8.0 |
| beta | 9.3 | 28.4 |
| kappa | 3.3 | 10.1 |
| Total Whey Proteins | 6.3 | 19.3 |
| alpha lactalbumin | 1.2 | 3.7 |
| beta lactoglobulin | 3.2 | 9.8 |
| BSA | 0.4 | 1.2 |
| Immunoglobulins | 0.7 | 2.1 |
| Proteose peptone | 0.8 | 2.4 |

The casein represents about 80% of milk proteins of milk [76, 199, 42, 117, 46]. The principal casein fractions are alpha(s1) and alpha(s2)-caseins, β-casein, and kappa-casein as shown in Tables 4 and 5. The common compositional factor is that caseins are conjugated proteins, most with phosphate group(s) esterified to serine residues. These phosphate groups are important to the structure of the casein micelle. Calcium binding by the individual caseins is proportional to the phosphate content. The conformation of caseins is much like that of denatured globular proteins. The high number of proline residues in caseins causes particular bending of the protein chain and inhibits the formation of close-packed, ordered secondary structures [76, 199, 42, 117, 46]. Caseins contain no disulfide bonds. As well, the lack of tertiary structure accounts for the stability of caseins against heat denaturation because there is very little structure to unfold. Without a tertiary structure there is considerable exposure of hydrophobic residues. This results in strong association reactions of the caseins and renders them insoluble in water. Within the group of caseins, there are several distinguishing features based on their charge distribution and sensitivity to calcium precipitation:
alpha(s1)-casein: (molecular weight 23,000; 199 residues, 17 proline residues)
Two hydrophobic regions, containing all the proline residues, separated by a polar region, which contains all but one of eight phosphate groups. It can be precipitated at very low levels of calcium.
   alpha(s2)-casein: (molecular weight 25,000; 207 residues, 10 prolines)
Concentrated negative charges near N-terminus and positive charges near C-terminus. It can also be precipitated at very low levels of calcium.
   β-casein: (molecular weight 24,000; 209 residues, 35 prolines)
Highly charged N-terminal region and a hydrophobic C-terminal region. Very amphiphilic protein acts like a detergent molecule. Self association is temperature dependant; will form a large polymer at 20° C. but not at 4° C. Less sensitive to calcium precipitation.
   kappa-casein: (molecular weight 19,000; 169 residues, 20 prolines)
Very resistant to calcium precipitation, stabilizing other caseins. Rennet cleavage at the Phe105-Met106 bond eliminates the stabilizing ability, leaving a hydrophobic portion, para-kappa-casein, and a hydrophilic portion called kappa-casein glycomacropeptide (GMP), or more accurately, caseinomacropeptide (CMP).

Most, but not all, of the casein proteins exist in a colloidal particle known as the casein micelle [76, 199, 42, 117, 46].

TABLE 5
Caseins Composition

| | |
|---|---|
| Fat (%) | 4.1 |
| Protein (%) | 3.6 |
| Protein/fat | 0.9 |
| Lactose (%) | 4.9 |
| 4-0-β-D-galactopyranosyl-D glucopyranose | |
| Ash (%) | 0.8 |
| Minerals and Vitamins | 0.2 |
| Total solids (%) | 15.0 |

Its biological function is to carry large amounts of highly insoluble CaP to mammalian young in liquid form and to form a clot in the stomach for more efficient nutrition. Besides casein protein, calcium and phosphate, the micelle also contains citrate, minor ions, lipase and plasmin enzymes, and entrapped milk serum [76, 199, 42, 117]. These micelles are rather porous structures, occupying about 4 ml/g and 6-12% of the total volume fraction of milk.

4.2.3 Agar

Agar is extracted from the red seaweeds belonging to the family Rhodophyceae, mainly from Gelidium and Gracilaria species. Agar is a polymer of agarobiose, a disaccharide composed of D-galactose and 3,6 anhydro-L-galactose linked in b-1,4. The link between 2 agarobiose units is in a-1,4. This polymer can be separated into two fractions: the pure disaccharide (agarose) and the less and more methylated, sulphated and pyruvated disaccharide (agaropectin). The agaropectin proportion and its substitution level are important as for the charge carried by the molecule—from which its behaviour and its affinity—, and as for the gel features in terms of strength, elasticity, fusion and gelation temperature. Generally speaking, a high substitution level gives a soft, flexible and elastic gel. Agar gelation in an aqueous solution is reversible. When hot, the polymer configures itself as a bundle. When the solution is cooling, the chains wrap together and join two by two by hydrogen links to form double helices giving then a three-dimensional network all the more solid because the number of hydrogen links is great. Agar is a neutral product from an organoleptic point of view: colourless, odourless, and tasteless. So, it does not interfere with the gustative features of the food in which it is incorporated. High gelling strength, usually 600 to 800 g/cm² and reaching sometimes 1100 g/cm² for a solution at 1.5%. Having an exceptional resistance, agars are the most resistant gels. Spontaneous gelation without adding anything, without medium modification. Reversibility: soluble in hot water, gelation temperature of agar is about 35° C., so it has undeniable advantages of use, this reversibility changing nothing of its properties.

5.1.1 Inhibition of Spores Using Glycoconjugates

The ultra-dishes Petri exposed to UV irradiation during 5 hrs to prevent cross contamination. Fabrication “simulated life environment” was prepared on UV irradiated ultra-dishes Petri. TSA was the main compound of our “simulated life environment” system. Detailed composition of TSA components has shown at 4.1.1 section. To prevent cross contamination the fabricated “simulated life environment” systems were exposed to UV irradiation overnight. Disaccharide-PAA-flu glycoconjugates, as shown in FIGS. 9-11, were prepared according manufacture instruction [63]. Than glycoconjugate solutions were diluted ×10, ×100, and ×1000 into a PBS/0.2% NaH₃ buffer. Spores handling experiments were carried out under a Biological Safety Cabinet Class IIA/B3 Form a Scientific Inc. (Maijetta, Ohio, USA). B. cereus and B. subtilis species possess a different morphology, as shown in FIGS. 12-14. It is known that B. subtilis has no more than an exosporium and B. cereus/B. anthracis have an appendages and exosporium [105, 38]. In addition, these spores exhibit different glycan structures and monosaccharides expression on the spore exterior, as shown in FIGS. 15 and 16. These monosaccharides created receptors network on the exterior surface of the spore. Briefly, bacterial spore suspensions, as shown in FIGS. 12-14, were vortex well. Than, 2, or 3, or 4 μL of B. cereus, B. subtilis spore suspensions have inoculated into sterile tubes (0.5 ml). Later on, prepared disaccharide-PAA-flu glycoconjugates 10 μL, as shown in FIG. 9-11, were added to each studied species including B. cereus, B. subtilis spores, and concentration (2, or 3, or 4 μL) separately to each tubes. Spore-glycoconjugate mixtures were vortex well during 30-40 sec. Later on, samples mixtures were incubated 1 hr at room temperature. The processing of the samples after spores inhibition using glycoconjugates treatment involved the following procedures. Tissue culture water 60 μL was added to inhibited spores in each tube and mixed well. The mixtures were serially diluted from 10⁻¹ to 10⁻⁷. Spore inhibition with glycoconjugates was confirmed by Atomic Force Microscopy (AFM) as well, as shown in FIGS. 7 and 8. Afterward, the mixtures were carefully plated onto fabricated “simulated life environment” on Petri dishes and incubated at 37° C. for 14-16 hrs. After the incubation, the resulting colony forming units (CFU) of remained spores were counted using software Quantity One 4.4.1 (BioRad Laboratories, Inc., Hercules, Calif.). The sum of the counted CFU from each set of serially diluted samples then compares with the control CFU provided by the supplier company to determine the inhibition efficacy of studied glycoconjugates. The x-axis represents glycoconjugates (native, ×10, ×100, ×1000 dilution) and the y-axis displays the log of the ratio of the number of viable spores remaining (N) to the CFU control number (N₀). The inhibition efficacy of the glycoconjugates on inhibition of B. cereus, as shown in FIGS. 4 and 5, and B. subtilis spores, as shown in FIGS. 2 and 3, demonstrated shown below. Dilution of glycoconjugates promotes inhibition, as shown in FIG. 20. Kinetics studies demonstrate that glycoconjugates can control germination of spores, as shown in FIG. 21. Then, we have compared the efficacy of two independent inhibition methods: chemical using glycoconjugates and physical using plasma torch. Spore inhibition using glycoconjugates is by far more effective than decontamination using the plasma torch alone, as shown in FIG. 21. Changes in the glycoconjugate concentration did not potentially hinder the recognition and inhibition of spores. Glycoconjugates were able to entrap bacterial spores, as shown in FIGS. 7 and 8.

6.1 Optical and Fluorescence Microscopy

Glycoconjugates, as shown in FIG. 9, were examined under a Nikon Eclipse E400 POL fluorescence microscope at a magnification of ×400, as shown in FIG. 2. Digital micrographs of glycoconjugates were acquired in real-time, as shown in FIG. 10.

7.1 Scanning Electron Microscopy (SEM)

Spore samples (10⁴ CFU) for SEM observations were deposited on mica disks and desiccated for 7 days. Samples were then coated with a 10 nm thin film of evaporated gold [16] for 60 sec and then examined with a Hitachi S-570 scanning electron microscope (Hitachi Ltd., Tokyo, Japan) at an accelerating voltage of 15-20 kV, as shown in FIG. 12.

8.1 Atomic Force Microscopy (AFM)

AFM were performed for glycoconjugate, as shown in FIG. 9, solutions of untreated spores (10⁴ CFU), as shown in FIGS. 13 and 14, and inhibited spores, as shown in FIGS. 7 and 8. Inhibition effects of glycoconjugate, as shown in FIGS. 10 and 11, on B. cereus, as shown in FIG. 7, and B. subtilis, as shown in FIG. 8, were confirmed using AFM as well. For instance, solutions of untreated spores (10⁴ CFU), as shown in FIGS. 13 and 14, were immobilized on mica discs [36] using sterile syringes, then dried in air at 20-25° C. Prepared samples were later mounted on an AFM sample holder for imaging. All AFM observations were carried out at 20° C., using a Nano Scope® IIIa controller as well as a MultiMode™ microscope (Digital Instruments, Inc., Santa Barbara, Calif.) operating in tapping mode (amplitude) together with an E-scanner. A 125-μm silicon Nanoprobe (Digital Instruments, Inc.) was also employed. The calculated spring constant was 0.3 N/m. The resonance frequency remained in the range of 240-280 kHz, and the scan rate was of 1 μm(s). Flattening and high-pass filtering of the image data were performed in order to remove the substrate slope from images as well as high-frequency noise strikes, otherwise more pronounced in the high-resolution tapping mode imaging.

9.1 Isolation of Bacterial Spores Exterior Layer

The isolation of the exterior layer and inner part of spores [120, 179] was performed as described earlier [105] with minor modification. Briefly, a 500 μL spore suspension with a concentration of 2×10⁶/100 μL was mixed with a 2% solution of 2-mercaptoethanol (1 mM carbonate-bicarbonate buffer, pH 10.0), vortexed for 5 min, and incubated at 37° C. for 120 min. After exposing it to the reagent, the mixture was centrifuged at 4,000×g for 20 min. Fractions containing appendages (supernatant) and spore inner constituents (pellets), were washed 4 times with deionized water. Successful isolation of fractions was confirmed by AFM prior to further use [179].

10.1 Fluorophore Assisted Carbohydrate Electrophoresis (FACE) of Bacterial Spores Exterior Layer

FACE was carried out with the FACE® monosaccharide composition and N-profiling kit according to manufacturer's instructions (Prozyme®, San Leandro, Calif.). The kit contains reagents and buffers required to hydrolyze oligosaccharides of intact glycoproteins and to analyze neutral and amine monosaccharides constituents such as AMAC-labeled N-acetylglucosamine (GlcNAc), N-acetylgalactosamine (GalNAc), galactose, mannose, fucose, and glucose, as shown in FIGS. 15 and 16, and analyze glycans profiling.

11.1 Spectroscopical Measurements

The germination rate of spores before and after inhibition during kinetics study [180]. The more spores germinate, the higher the turbidity and optical density (OD) was. Spectrophotometry of spore suspension prior and after treatment was carried out with a SpectraMax® Plus³⁸⁴ microplate reader (Molecular Devices Corp., Sunnyvale, Calif.) at 600 nm. Time and temperature effects have determined in the same manner. Inhibition efficiency after exposure was compared with controls and plotted. The sum of observed OD from each set of experiment was compare with untreated controls to determine inhibition efficacy.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### LIST OF REFERENCES

1. Alasri et al., Can. J. Microbiol 1993: 39: 52-60.
2. Alves A. P., Mulloy B., Moy G. W., Vacquier V. D., and Mourao P. A. Glycobiology 1998; 8: 939-946.
3. Anderson P. W., Pichichero, M. E., Stein, E. C., Porcalli, S., Betts, R. F., Connuck, D. M., Lorones, D., Insel, R. A., Zahradnik, J. M., and Eby, R. J. Immunol. 1989; 142: 2464-2468.
4. Anon. Surveillance for adverse events associated with anthrax vaccination. U.S. Department of Defense, 1998-2000. MMWR 2000; 49(16):341-5.
5. Asano M., K. Furukawa, M. Kido, S. Matsumoto, Y. Umesaki, N. Kochibe, and Y. Iwakura. 1997. EMBO J. 1997; 16: 1850-1857.
6. Austrup F., Vestweber D., Borges E., Lohning M., Brauer R., Herz U., Renz H., Hallmann R., Scheffold A., Radbruch A., Hamann A. Nature 1997; 385: 81-83.
7. Baillie L., and Read T. D., Curr. Opin. Microbiol. 2001; 4, 78.
8. Baillie L., Townend T., Walker N., Eriksson U., Williamson D. FEMS Immunology and Medical Microbiology 2004; 42: 267-270.
9. Baillie L. J. Appl. Microbiol. 2001; 91: 609-13.
10. Bailey C. P., and von Holy A., Food. Microbiol. 1993; 10: 287.
11. Baize J. Global soybean meal analysis project. Conducted for the Quality Committee of the United Soybean Board. 1999. John C. Baize and Associates, 7124 Carol Lane, Falls Church, Va. 22042-3714.
12. Bartlett A. L., and K. K. Atherosclerosis 1998; 138: 237-245.
13. Baum L. G., Derbin K., Perillo N. L., Wu T., Pang M., and Uittenbogaart C. J. Biol. Chem. 1996; 271: 10793-10799.
14. BD Biopharmaceutical Production. Bionutrient technical manual. 2004.
15. Beauchamp et al., Crit. Rev. Toxicol. 1992; 22:143-174.
16. Bergeret, G.; Gallezot, P. In Handbook of heterogeneous catalysis. vol. 2 Ertl, G., Knözinger, H., Weitkamp, J. Eds, Wiley-VCH, Weinheim, Germany. [1997].
17. Bhalla D. K., and Warheit D. B., Toxicology and Applied Pharmacology, 2004; 199:71.
18. Bhavanandan V. P. Glycobiology 1991; 1: 493-503.
19. Blum A. S., and Barnstable C. J. PNAS 1987; 84: 8716-8720.
20. Borman S. C&EN, 2004; Aug. 9: 31-35.
21. Bovin, N. V. In Glycosciences: Status and Perspectives. Gabius H. J., and Gabius S., Eds; Chapman & Hall, London, 1997, pp. 277-289.
22. Bovin, N. V. Glycocon. J., 1998; 15: 431-446.
23. Britis P. A., Canning D. R., Silver J. Science 1992; 255: 733-736.
24. Carpo M., Pedotti R., Lolli F., et al. J. Neuro. Sci. 1998; 155: 186-191.
25. Charest P. M., Roth J. PNAS 1985; 82: 8505-8512.
26. Chen L.-Ch., Carley K. M., Fridsman D., Kaminsky B., and Yahja A., Decision Support Systems, 2004; 12, August (on-line).
27. Cornil J. Cell Biol. 1990; 111: 773-781.
28. Curry, A. S., Joyce G. G., and McEwen G. N. Jr. CTFA Microbiology guidelines. The Cosmetic, Toiletry, and Fragrance Association, Inc. Washington, D.C. 1993.
29. Dragon and Rennie Can. Vet. J. 1995; 36:295-301.
30. Debrauwére H, Buard J, Tessier J, Aubert D, Vergnaud G, Nicolas A. Nat. Genet. 1999; 23: 367-71.
31. De Bolle X, Bayliss C D, Field D, van de Ven T, Saunders N J, Hood D W, Moxon E R:. Mol. Microbiol. 2000; 35: 211-22.
32. Dennis CRC Press 1992; 161-194.
33. Dennis Nature 1982; 300: 274-276.
34. de Vos V., Proceedings of the International Workshop on Anthrax, Winchester, UK, 11-13 Apr. 1989. Salisbury Med. Bull. 1990; 68 (Suppl.): 19.
35. Dietz P., Bohm R., and Strauch D., Zbl Bakt Hyg 1980; 171:158.
36. Digital Instruments, Inc., Manuals. Santa Barbara, Calif.
37. Drickamer et al., Annu. Rev of Cell Biol. 1993; 9: 237-264.
38. Driks A. Microbiol. Molecul. Biol. Rev. 1999; 63: 1-20.
39. Dohring et al., Crti. Rev Immunol. 17: 285-299.
40. Drobniewski Clin. Microbiol. Rev. 1993; 6:324-338.
41. Duesbery N. S., Vande Woude G. F., Anthrax toxins, Cell. Mol. Life. Sci. 1999; 55:1599-1609.
42. Dziuba J., Babuchowski A., Smoczynski M., and Smietana Z. 1999. Fractal analysis of caseinate structure. Int. Dairy J. 9:287-292.
43. Hess et al., Amer. J. Dent. 1991; 4: 51-56.
44. Hills, J. Gen Microbiol 4:38, 1950.
45. Human Nutrition Service, U.S. Department of Agriculture. 1986. Composition of foods: legume and legume products. Agriculture handbook, No. 8-16, revised. U.S. Department of Agriculture, Washington, D.C.
46. http://arbl.cvmbs.colostate.edu/hbooks/pathphys/digestion/pancreas/exocrine.html
47. Ebedes H., Madoqua 1976; 10: 99.
48. Feizi Nature 1985; 314: 54-57.
49. Fenderson, Bioassays 1990; 12:173-179.
50. Franz et al., JAMA 1997; 278: 399-411.
51. Fredman Neurochemistry Research 1997; 22: 1071-1083.
52. Fitchette-Laine Plant Journal 1997; 12: 1411-1417.
53. Friedlander A. M. J. Biol. Chem. 1986; 261: 7123-7126.
54. Fuhlbrigge Nature 1997; 389: 978-981.
55. Fukuda Cancer Research 1996; 56: 2237-2244.
56. Gabius Glycoscience. 1997.
57. Galanina, O.; Feofanov, A.; Tuzikov, A. B.; Papoport, E.; Crocker, P. R.; Grichine, A.; Egret-Charlier, M.; Vigny, P.; Le Pendu, J.; Bovin, N. V. Spectrochimica Acta Part A—Molecular and Biomolecular Spectroscopy. 2001; 57: 2285-2296.
58. Gerhardt P., and Ribi E., J. Bacteriol. 1964; 88: 1774.
59. Greenberg, A. E., Clesceri L. S., and. Eaton A. D (eds). Standard methods for the examination of water and wastewater, 19th ed. American Public Health Association, Washington, D.C. 1995.
60. Gladstone G. P. Br. J. Exp. Pathol. 1946; 27: 394-418.
61. Giammona et al. Anticancer Agent Coupled to Polyaspartamide as a Drug Carrier. Eur. J. Pharm. Biopharm, 1992; 38: 159-162.
62. Giammona et al. Int. J. Pharmaceutics. 1989; 57: 55-62.
63. GlycoTech Inc., Rockville, Md. Instruction manual. 2004
64. Gridley et al., Trends in Genetics 1987; 3: 162-166.
65. Griffiths M. W., and Phillips J. D., J. Soc. Dairy Technol. 1990; 43: 62.
66. Goedert Nature 1997; 383: 550-553.
67. Gruchalla R. S., and Jones J., J. Allergy Clin. Immunol., 2003; 112: 675.
68. Hahn Ann. Rev. Phytopath. 1996; 34: 387-412.
69. Hakomori Cancer Research 1996; 56: 5309-5318.
70. Hambleton P., Carman J. A., Melling J. Vaccine. 1984; 2 (2):125-32.
71. Hambleton P., Turnbull P. C. B., Adv. Biotechnol. Processes 1990; 13: 105-122.
72. Harris S. et al., Proc. Natl. Acad. Sci. 1997; 94, 6: 2454-2459
73. Hitata Am. J. Resp. Cell Mol. Biol. 1998; 18: 12-20.
74. Hennet, et al., PNAS 1998; 95: 4504-4509.
75. Hoess R. et al, Gene 1993; 128: 43.
76. Huffman L. M. and Harper, W. J. Symposium: Marketing dairy value through technology: maximizing the value of milk through separation technologies. J. Dairy Sci. 1999; 82:2238-2244.
77. Hugh-Jones M., J. Appl. Microbiol. 1999; 87:189.
78. Hunter L., W. Corbett, and C. Grindem, J. Am. Vet. Med. Assoc. 1989; 8: 1028
79. http://www.soymeal.org
80. http://www.scientificpsychic.com/fitness/carbohydrates.html
81. Jackson et al., PNAS 1998; 95:1224-1229.
82. Jackson P. J., Hill K. K., Pardington P. E., and Ticknor L. O., In: Program and abstracts book of 4^{th} International conference of Anthrax, Annapolis, Md., USA, Jun. 10-13: 15, 2001.
83. Jacobs J. Infect. Dis. 1997; 175: 729-733.
84. Jamie W. E. Primary Care Update for OB/GYNS 2002; 9: 117-121.
85. Joellenbeck L M, Zwanziger L L, Durch J S, Strom B L. Washington, D.C.: National Academy Press; 2002.
86. Ilver Science 1998; 279: 373-377.
87. Imundo PNAS 1995; 92: 3019-3023.
88. Inglesby T. V., O'Toole T., Henderson D. A., Bartlett J. G., Ascher M. S., Eitzen E., Friedlander A. M., Gerberding J., Hauer J., Hughes J., McDade J., Osterholm M. T., Parker G., Perl T. M., Russell Ph. K., Tonat K., JAMA 2002; 287:2236.
89. Ivins et al., Vaccine 1995; 13: 1779-1784.
90. Kacack M. B. et al., Infec. Immun. 1991; 61: 2289-.
91. Kahn Infectious Immunity 1996; 64: 2649-2656.
92. Kalchayanand N., Ray B., Field R. A., and Johnson M. C., J. Food Prot. 1989; 52: 424.
93. Kalchayanand N., Ray B., and Field R. A., J. Food Prot. 1993; 56: 13.
94. Karlsson Curr. Opin. Struct. Biol. 1995; 5: 622-635.
95. Karlsson Annu. Rev. Biochem 1989; 58: 309-350.
96. Kasper D. et al., J. Clin. Invest. 1996; 98, 10: 2308-2314.
97. Khan Mol. Glycobiology. 1994; 206-223.
98. Kewitz Glycoconjugate Journal 1995; 12: 714-720.
99. King Clinic North American 1996; 25: 255-270.
100. Kiss Curr. Opin. Neurobiol. 1997; 7: 640-646.
101. Kitagawa J. Biol. Chem. 1994; 269: 17872-17878.
102. Korbata Glycobiology 1991; 1: 5-8.
103. Komfeld Annu. Rev. Biochem. 1985; 54: 631-664.
104. Kopecjek J., Duncan R., J. J. Controlled Release. 1987; 6: pp. 315-327.
105. Kozuka, S.; Tochikubo, K. Microbiol. Immunol. 1985; 29: 21-37.
106. Kyriacou D. N., Stein A. C., Yarnold P. R., Courtney D. M., Nelson R. R., Noskin G. A., Handler J. A., and Frerichs R. R., The Lancet, 2004; 364: 9432, 449.
107. Kunitz, M. Science 1945; 101: 668-669.
108. Kukuruzinska et al., Annu. Rev. Biochem 1987; 56: 915-944.
109. Kuwabara Trends in Genetics 1997; 13: 455-460.
110. Lawson P., Dainty R. H., N. Kristiansen, J. Berg, and M. D. Collins, Lett. Appl. Microbiol. 1994; 19: 153.
111. Laine Glycobiology 1994; 4: 1017-1023.
112. Lasky Science 1992; 258: 964-969.
113. Leavitt, J. M., Naidorf I. J. and Shugaevsky P. The NY J. Dentist. 1955; 25: 377-382.
114. Lee New England J. of Medicine 1991; 324: 1084-1090.
115. Lees et al., J. Med. Chem. 1994; 37: 3419-3433.
116. Leffler Trends in Glycosci Glycotechnol. 1997; 9: 31-40.
117. Lehninger A. L. 1975. Proteins: covalent backbone and amino acid sequence. In Biochemistry. 2nd ed. Worth Publishers, Inc., New York.
118. Lesley Glycoconjugate 1997; 14: 611-622.
119. Lerouge Nature 1990; 344: 781-784.
120. Levon K., O. Tarasenko, and Bin Yu. Glycoconjugate Sensors (November, 2003) U.S. patent application Ser. No. 10/719,688, filed: Nov. 21, 2003; PCT Application: PCT/US03/37401 filed: Nov. 21, 2003.
121. Liener I. E., Sharon, N., Goldstein, I. J. eds The Lectins: Properties, functions and applications in biology and medicine. New York: Academic. 1986.
122. Lis European Journal of Biochemistry 1993; 218: 1-27.
123. Little S. F., and Ivins B. E., Microbes. Infect. 1999; 2, 131.
124. Little S. F., Ivins B. E., Fellows P. F., Friedlander A. M. Infect. Immunol. 1997; 65: 5171-5175.
125. Little S. F., Webster W. M., Ivins B. E., Fellows P. F., Norris S. L., Andrews G. P. Vaccine. 2004; 22: 2843-2852.
126. Lineaweaver et al., Arch. Surg. 1985; 120: 267-270.
127. Lobsanov, et al., Trends in Glycosci Glycotechnol. 1997; 9: 145-154.
128. Lockey D., Davies G., Resuscitation. 2003; 58: 293.
129. Lowe J. Clin. Invest. 1997; 99: 822-826.
130. Maekawa J. Clin. Invest. 1992; 990: 67-76.
131. Manafo W. J. et al., J. Immunol. 1987; 139: 2702-.
132. Martin E. W., Remington's Pharmaceutical Sciences.
133. Mawhinney Carbohydrate Research 1992; 235: 179-197.
134. McIntyre S., Ikawa J. Y., Parkinson N., Haglund J., and Lee J., J. Food Prot. 1995; 58: 319.
135. McNamara et al., Science 1984; 226: 1325-.
136. Meer R. R., Baker J., Bodyfelt F. W., and Griffiths M. W., J. Food Prot. 1991; 54: 969.
137. Mellors Trends in Biotechnology 1994; 12:15-18.
138. Mengeling Curr. Opin. Struct. Biol. 1998; 8: 572-577.
139. Meselson et al. Science 1994; 266:1202-1208.
140. Miller Nature 1992; 357: 589-593.
141. Moens et al., Arch. Microbiol. 1997; 168: 169-175.
142. Mobley Military Med. 1995; 160: 547-553.
143. Montreuil Glycoproteins and Disease. 1996.
144. Morgan Toxicol. Pathol. 1997; 25: 291-307.
145. Nakamori Dis. Colon Rectum 1997; 40: 420-431.
146. Oldenberg, K. R., Proc. Nat. Acad. Sci. USA 1992; 89: 5393.
147. Orth, D. S. Handbook of cosmetic microbiology. Marcel Dekker, Inc., New York, N.Y. 1993.
148. Palic Trends in Glycoscience and Glycotechnology 1996; 8: 37-49.
149. Parekh Nature 1985; 316: 452-457.
150. Paulsson J. C. In: The Receptors (Vol. 2) (ed. P. M. Conn), Academic Press, New York, pp. 131-219, 1985.
151. Philips Science 1990; 250: 1130-1132.
152. Pile et al. Arch. Intem. Med. 1998; 158: 429-434.
153. Puziss M., Manning L. C., Lynch L. W., Barclay E., Abelow I., Wright G. G. Appl. Microbiol. 1963; 11: 330-334.
154. Quantity One 4.4.1″ software manual/BioRad Laboratories, Inc., Hercules, Calif.
155. Radnedge L., Agron P. G., Hill K. K., Jackson P. J., Ticknor L. O., Keim P., and Andersen G. L., Appl. Environ. Microbiol. 2003; 69: 2755.
156. Raetz J. Bacteriology 1993; 175: 5745-5753.
157. Ramirez D. M., Leppla S. H., Schneerson R., Shiloach J. J. Ind. Microbiol. Biotechnol. 2002; 28: 232-238.
158. Raven Biological Lab. Instruction manual, 2004.
159. Reid Histochemical Journal. 1984; 16: 235-251.
160. Remington's Pharmaceutical Sciences, 1980. Mack Publishing Co., Easton, Pa., Arthur Osol (ed), 16.sup.th Edition; also see each of the later editions of the same (i.e., each later edition to date of Remington's Pharmaceutical Sciences)
161. Roberts Annual review of microbiology 1996; 50: 285-315.
162. Rohrig Science 1995; 269: 841-843.
163. Read T. D., Peterson S. N., Tourasse N., Baillie L. W., Paulsen I. T., Nelson K. E., Tettelin H., Fouts D. E., Eisen J. A., Gill S. R., Holtzapple E. K., Okstad O. A., Helgason E., Rilstone J., Wu M., Kolonay J. F., Beanan M. J., Dodson R. J., Brinkac L. M., Gwinn M., DeBoy R. T., Madpu R., Daugherty S. C., Durkin A. S., Haft D. H., Nelson W. C., Peterson J. D., Pop M., Khouri H. M., Radune D., Benton J. L., Mahamoud Y., Jiang L., Hance I. R., Weidman J. F., Berry K. J., Plaut R. D., Wolf A. M., Watkins K. L., Nierman W. C., Hazen A., Cline R., Redmond C., Thwaite J. E., White O., Salzberg S. L., Thomason B., Friedlander A. M., Koehler T. M., Hanna P. C., Kolsto A. B., and Fraser C. M., Nature. 2003; 423: 81.
164. Rogers P., Whitby S., and Dando M., Sci. Am. 1999; 280: 70.
165. Rotz L., Khan A., Lillibridge S., Ostroff S., and Hughes J., Emerg. Infect. Dis. 2002; 8: 225.
166. Russel Clin. Micro 1990; 3: 99-119.
167. Setlow P., Annu. Rev. Microbiol. 1995; 49: 29.
168. Setlow P., J. Appl. Bacteriol. 1994; 176 (Symp. Suppl.):49S.
169. Schmidt 5^{th} edition, p. 2 Wm C. Brown, New York, 1996.
170. Schmidt Foundations of Parasitology. 5^{th} edition 1996.
171. Schneerson R. et al., Inf. Immun. 1986; 52: 519-.
172. Schreiber et al, J. Inf. Dis. 1991; 164: 507.
173. Showalter Plant Cell 1993; 5: 923.
174. Stahl et al., Curr. Opin. Immunol. 1998; 10: 50-55.
175. Stanley J. L., Smith H. J. Gen. Microbiol. 1961; 26: 49-66.
176. Springer Annu. Rev. Physiol. 1995; 57: 827-872.
177. Springer Nature 1991; 349: 196-197.
178. Sousa J. C., Silva M. T., and Balassa G., Nature 1976; 263: 53.
179. Tarasenko O., Islam Sh., Paquiot D., and Levon K. Carbohydrate Research. 2004; 339: 2859-2870
180. Tarasenko O., Paquiot D., Islam Sh, Alusta P., Levon K. Inhibition of sporeforming pathogens using glycoconjugates. Section: ASBMB/ASPET: Experimental Biology. Annual Meeting and the XXXV International Congress of Physiological Sciences in San Diego, Calif. Mar. 31-Apr. 6, 2005.
181. Tarasenko O., Paquiot D., Islam Sh, Alusta P., Levon K. Recognition and inhibition of Bacillus species spores “Bacterial Spores: Challenges and Future Directions for Biodefense Meeting” Argonne National Laboratory, Chicago Ill., Nov. 14-16, 2004.
182. Taylor-Papadimitriou Immunology Today 1997; 18: 105-107.
183. Taniguchi Glycobiology 1996; 6: 691-694.
184. Thompson, Glycoconjugate 1997; 14: 837-845.
185. Turnbull P. C. B., In: Principles of Bacteriology, Virology and Immunity, 8th Edition, Arnold, London. 1990; 365.
186. Turnbull P. C. B. Vaccine. 1991; 9 (8):533-9.
187. Turnbull P. C. B., Hutson R. A., Ward M. J., Jones M. N., Quinn C. P., Finnie N. J., Duggleby C. J., Kramer J. M., Melling J., J. Appl. Bacteriol. 1992; 72: 21.
188. Turnbull P. C. B., In: Principles of Bacteriology, Virology and Immunity, 8th Edition, Arnold, London. 1990; 365. Holubik C. A., Tomlinson S. R., Advances in Health Care Management 2004; 4: 287.
189. Turnbull P. C. B. Curr. Opin. Infect. Dis. 2000; 13 (13):113-20.
190. Turnbull P., Carman J., Lindeque D., Joubert F., Hubschle O., and Snoeyenbos G., Madoqua 1989; 16: 93.
191. United States Pharmacopeial Convention, Inc. 2004. United States Pharmacopeial Convention, Inc., Rockville, Md.
192. U.S. Food and Drug Administration. 1995. Bacteriological analytical manual, 8^{th} ed., AOAC International, Gaithersburg, Md.
193. U.S. Pat. No. 6,517,827.
194. U.S. Pat. No. 5,971,511.
195. U.S. Pat. No. 5,576,305.
196. U.S. Pat. No. 5,374,655.
197. U.S. Pat. No. 5,453,272.
198. Valuation P. et al., J. Mol. Biol. 1996; 261: 11-22.
199. Van Boekel. Int. Dairy. J. 1999; 9: 237-241.
200. Van der Eijnden, Curr. Opin. Struct. Biol. 1993; 3: 711-721.
201. Vergnaud G., Denoeud F. Genome Res. 2000; 10: 899-907.
202. Vlassara Diabetes 1996; 45: 65-66.
203. VonItzstein et al. Curr. Med. Chem. 1996; 4: 185-210.
204. Voet Biochemie, VHC 1992.
205. Weis et al., Immunol. Rev. 1998; 163: 19-34.
206. Welkos et al., Infect. Immun. 1986; 51: 795-800.
207. Welkos and Friedlander
208. Microb. Path. 1988; 4: 53-69.
209. Westeruik et al., Proc. Nat. Acad. Sci. USA 1995; 92: 4021-4025.
210. Wilson J., and Russel K., J. Bacteriol. 1964; 87, 237.
211. Wong Angew. Chem. Intl. Ed. Engl. 1995; 34: 412-432.
212. Zaman Handbook of Medical Parasitology, 2^{nd} edition. 1989.
213. Zhang et al., 1992; 69: 861-869.
214. Zopf Lancet 1996; 347: 1017-1021.
215. Zorc B. et al., 1993; 99: 135-143.
216. Zorc B. and Butula, I. Acta Pharm. 1994; 44: 103-108.
217. Yamashita, Cancer Research 1995; 55: 1675-1679.
218. Youakim J. Cell Biol. 1994; 126: 1573-1583.

1. (canceled)

2. (canceled)

3. (canceled)

4. (canceled)

5. (canceled)

6. A pharmaceutical composition comprising:
a. at least one glycoconjugate having at least one disaccharide moiety or higher polysaccharide; and
b. a pharmaceutically acceptable carrier.

7. The pharmaceutical composition of claim 6, wherein the glycoconjugate is a molecule containing a carbohydrate moiety that is glycosylated.

8. The pharmaceutical composition of claim 7, wherein the glycosylated carbohydrate moiety comprises a glycoprotein or a glycolipid.

9. The pharmaceutical composition of claim 6, wherein the pharmaceutically acceptable carrier comprises at least one of a polymer, a glycoconjugate, water, oils, saline, a glycerol solution and any mixture thereof.

10. An inhibitor for inhibiting spores of Bacteria genus, comprising at least one glycoconjugate having at least one disaccharide moiety or higher polysaccharide.

11. The inhibitor of claim 10, wherein the glycoconjugate is a molecule containing a carbohydrate moiety that is glycosylated.

12. The inhibitor of claim 11, wherein the glycosylated carbohydrate moiety comprises a glycoprotein or a glycolipid.

13. The inhibitor of claim 10, wherein the Bacteria genus includes B. cereus, B. thuringiensis, B. pumilus, and B. subtilis.

14. (canceled)

15. (canceled)

16. (canceled)

17. (canceled)

18. A pharmaceutical composition comprising:
a. at least one glycoconjugate having at least one disaccharide moiety or higher polysaccharide; and
b. a pharmaceutically acceptable carrier.

19. The pharmaceutical composition of claim 18, wherein the glycoconjugate is a molecule containing a carbohydrate moiety that is glycosylated.

20. The pharmaceutical composition of claim 19, wherein the glycosylated carbohydrate moiety comprises a glycoprotein or a glycolipid.

21. The pharmaceutical composition of claim 18, wherein the pharmaceutically acceptable carrier comprises at least one of a polymer, a glycoconjugate, water, oils, saline, a glycerol solution and any mixture thereof.

22. An inhibitor of sporeforming pathogens or agents, comprising at least one glycoconjugate having at least one disaccharide moiety or higher polysaccharide.

23. The inhibitor of claim 22, wherein the glycoconjugate is a molecule containing a carbohydrate moiety that is glycosylated.

24. The inhibitor of claim 23, wherein the glycosylated carbohydrate moiety comprises a glycoprotein or a glycolipid.

25. (canceled)

26. (canceled)

27. (canceled)

28. (canceled)

29. (canceled)

30. (canceled)

31. An inhibitor of sporeforming pathogens or agents, comprising at least one glycoconjugate that is bondable to a sporeforming pathogen or agent.

32. The inhibitor of claim 31, wherein the glycoconjugate is a molecule containing a carbohydrate moiety that is glycosylated.

33. The inhibitor of claim 32, wherein the glycosylated carbohydrate moiety comprises a glycoprotein or a glycolipid.

34. The inhibitor of claim 32, wherein the glycosylated carbohydrate moiety comprises at least one disaccharide moiety or higher polysaccharide.

35. The inhibitor of claim 32, wherein the glycosylated carbohydrate moiety comprises a plurality of copies of same monosaccharide units.

36. The inhibitor of claim 32, wherein the glycosylated carbohydrate moiety comprises a plurality of copies of copies of different monosaccharide units.
